SEQUENCE LISTING (37 sequences)

SEQ ID NO: 1 - protein, 9 aa, Homo sapiens
YALNHTLSV

SEQ ID NO: 2 - protein, 9 aa, Homo sapiens
APALPGPQF

SEQ ID NO: 3 - protein, 9 aa, Homo sapiens
APAPTAVVL

SEQ ID NO: 4 - protein, 9 aa, Homo sapiens
APARPYQGV

SEQ ID NO: 5 - protein, 9 aa, Homo sapiens
LPHQPLATY

SEQ ID NO: 6 - protein, 6 aa, Homo sapiens
SSVSVY

SEQ ID NO: 7 - DNA, 18 bp, Homo sapiens
tcgtctgtttcagtgtat

SEQ ID NO: 8 - DNA, 18 bp, Homo sapiens
agcagcgtgagcgtgtac

SEQ ID NO: 9 - protein, 8 aa, Homo sapiens
YLSGSTLV

SEQ ID NO: 10 - DNA, 24 bp, Homo sapiens
tatttatcaggatccaccctggtt

SEQ ID NO: 11 - DNA, 24 bp, Homo sapiens
tacctgagcgggagcacactggtg

SEQ ID NO: 12 - protein, 18 aa, Homo sapiens
CAVKVSNAGGTSYGKLTF

SEQ ID NO: 13 - DNA, 54 bp, Homo sapiens
tgtgctgtgaaggtgtctaacgctggtggtactagctatggaaagctgacattt

SEQ ID NO: 14 - DNA, 54 bp, Homo sapiens
tgcgccgtgaaggttagtaacgccggcggcactagctacggaaagttgaccttc

SEQ ID NO: 15 - protein, 5 aa, Homo sapiens
LNHDA

SEQ ID NO: 16 - DNA, 15 bp, Homo sapiens
ttgaaccacgatgcc

SEQ ID NO: 17 - DNA, 15 bp, Homo sapiens
ctgaaccacgatgcc

SEQ ID NO: 18 - protein, 6 aa, Homo sapiens
SQIVND

SEQ ID NO: 19 - DNA, 18 bp, Homo sapiens
tcacagatagtaaatgac

SEQ ID NO: 20 - DNA, 18 bp, Homo sapiens
agtcagattgtgaacgat

SEQ ID NO: 21 - protein, 14 aa, Homo sapiens
CASSIAQGADTQYF

SEQ ID NO: 22 - DNA, 42 bp, Homo sapiens
tgtgccagtagtattgctcagggtgcagatacgcagtatttt

SEQ ID NO: 23 - DNA, 42 bp, Homo sapiens
tgcgctagcagcattgctcagggcgctgatacacagtacttt

SEQ ID NO: 24 - protein, 137 aa, Homo sapiens
MLLLLVPAFQVIFTLGGTRAQSVTQLDSQVPVFEEAPVELRCNYSSSVSVYLFWYVQYPNQGLQLLLKYLSGSTLVESINGFEAEFNKSQTSFHLRKPSVHISDTAEYFCAVKVSNAGGTSYGKLTFGQGTILTVHP

SEQ ID NO: 25 - DNA, 411 bp, Homo sapiens
atgctcctgctgctcgtcccagcgttccaggtgatttttaccctgggaggaaccagagcccagtctgtgacccagcttgacagccaagtccctgtctttgaagaagcccctgtggagctgaggtgcaactactcatcgtctgtttcagtgtatctcttctggtatgtgcaataccccaaccaaggactccagcttctcctgaagtatttatcaggatccaccctggttgaaagcatcaacggttttgaggctgaatttaacaagagtcaaacttccttccacttgaggaaaccctcagtccatataagcgacacggctgagtacttctgtgctgtgaaggtgtctaacgctggtggtactagctatggaaagctgacatttggacaagggaccatcttgactgtccatcca

SEQ ID NO: 26 - DNA, 411 bp, Homo sapiens
atgctgctgctgctggtgcccgccttccaggtgatcttcaccctgggcggcacccgggcccagagcgtgacacagctggatagccaggtgcccgtgttcgaggaggcccccgtggagctgcggtgcaactacagcagcagcgtgagcgtgtacctgttctggtacgtgcagtaccccaaccagggactgcagctgctgctgaagtacctgagcgggagcacactggtggagagcattaacgggtttgaagctgagttcaacaaatcccagacatcttttcacctgaggaagccaagcgtgcacatttccgacaccgccgagtacttctgcgccgtgaaggttagtaacgccggcggcactagctacggaaagttgaccttcggacaggggacaatcctgactgtccatccc

SEQ ID NO: 27 - protein, 132 aa, Homo sapiens
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIAQGADTQYFGPGTRLTVL

SEQ ID NO: 28 - DNA, 396 bp, Homo sapiens
atgagcaaccaggtgctctgctgtgtggtcctttgtttcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagtattgctcagggtgcagatacgcagtattttggcccaggcacccggctgacagtgctc

SEQ ID NO: 29 - DNA, 396 bp, Homo sapiens
atgagcaaccaggtgctgtgctgcgtggtgctgtgctttcttggcgctaacacagtggatggaggcattacacagagcccaaagtacctgtttagaaaggaggggcagaacgtgacactgagctgtgagcagaacctgaaccacgatgccatgtactggtacagacaagatccaggacaggggctgagactgatctactacagtcagattgtgaacgattttcagaagggagatattgccgagggctacagcgtgtctagggagaagaaggagtcttttccactgacagtgacttcagcccagaagaaccctacagccttttacctgtgcgctagcagcattgctcagggcgctgatacacagtactttggacctgggacaaggctgacagtgctg

SEQ ID NO: 30 - protein, 278 aa, Homo sapiens
MLLLLVPAFQVIFTLGGTRAQSVTQLDSQVPVFEEAPVELRCNYSSSVSVYLFWYVQYPNQGLQLLLKYLSGSTLVESINGFEAEFNKSQTSFHLRKPSVHISDTAEYFCAVKVSNAGGTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 31 - protein, 274 aa, Artificial Sequence
  amino acid sequence for Valpha (VJ) domain of TCR 1C5.6 and constant domain (murine)
MLLLLVPAFQVIFTLGGTRAQSVTQLDSQVPVFEEAPVELRCNYSSSVSVYLFWYVQYPNQGLQLLLKYLSGSTLVESINGFEAEFNKSQTSFHLRKPSVHISDTAEYFCAVKVSNAGGTSYGKLTFGQGTILTVHPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 32 - DNA, 837 bp, Homo sapiens
atgctcctgctgctcgtcccagcgttccaggtgatttttaccctgggaggaaccagagcccagtctgtgacccagcttgacagccaagtccctgtctttgaagaagcccctgtggagctgaggtgcaactactcatcgtctgtttcagtgtatctcttctggtatgtgcaataccccaaccaaggactccagcttctcctgaagtatttatcaggatccaccctggttgaaagcatcaacggttttgaggctgaatttaacaagagtcaaacttccttccacttgaggaaaccctcagtccatataagcgacacggctgagtacttctgtgctgtgaaggtgtctaacgctggtggtactagctatggaaagctgacatttggacaagggaccatcttgactgtccatccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtccagctga

SEQ ID NO: 33 - DNA, 825 bp, Artificial Sequence
  codon optimized nucleic acid sequence for Valpha (VJ) domain of TCR 1C5.6 and constant domain (murine)
atgctgctgctgctggtgcccgccttccaggtgatcttcaccctgggcggcacccgggcccagagcgtgacacagctggatagccaggtgcccgtgttcgaggaggcccccgtggagctgcggtgcaactacagcagcagcgtgagcgtgtacctgttctggtacgtgcagtaccccaaccagggactgcagctgctgctgaagtacctgagcgggagcacactggtggagagcattaacgggtttgaagctgagttcaacaaatcccagacatcttttcacctgaggaagccaagcgtgcacatttccgacaccgccgagtacttctgcgccgtgaaggttagtaacgccggcggcactagctacggaaagttgaccttcggacaggggacaatcctgactgtccatcccgacattcagaacccggaaccggctgtataccagctgaaggacccccgatctcaggatagtactctgtgcctgttcaccgactttgatagtcagatcaatgtgcctaaaaccatggaatccggaacttttattaccgacaagtgcgtgctggatatgaaagccatggacagtaagtcaaacggcgccatcgcttggagcaatcagacatccttcacttgccaggatatcttcaaggagaccaacgcaacatacccatcctctgacgtgccctgtgatgccaccctgacagagaagtctttcgaaacagacatgaacctgaattttcagaatctgagcgtgatgggcctgagaatcctgctgctgaaggtcgctgggtttaatctgctgatgacactgcggctgtggtcctcatga

SEQ ID NO: 34 - protein, 309 aa, Homo sapiens
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIAQGADTQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 35 - protein, 305 aa, Artificial Sequence
  amino acid sequence for Vbeta (VDJ) domain of TCR 1C5.6 and constant domain (murine)
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIAQGADTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 36 - DNA, 930 bp, Homo sapiens
atgagcaaccaggtgctctgctgtgtggtcctttgtttcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagtattgctcagggtgcagatacgcagtattttggcccaggcacccggctgacagtgctcgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 37 - DNA, 918 bp, Artificial Sequence
  codon optimized nucleic acid sequence for Vbeta (VDJ) domain of TCR 1C5.6 and constant domain (murine)
atgagcaaccaggtgctgtgctgcgtggtgctgtgctttcttggcgctaacacagtggatggaggcattacacagagcccaaagtacctgtttagaaaggaggggcagaacgtgacactgagctgtgagcagaacctgaaccacgatgccatgtactggtacagacaagatccaggacaggggctgagactgatctactacagtcagattgtgaacgattttcagaagggagatattgccgagggctacagcgtgtctagggagaagaaggagtcttttccactgacagtgacttcagcccagaagaaccctacagccttttacctgtgcgctagcagcattgctcagggcgctgatacacagtactttggacctgggacaaggctgacagtgctggaagatctacgtaacgtgacaccacccaaagtctcactgtttgagcctagcaaggcagaaattgccaacaagcagaaggccaccctggtgtgcctggcaagagggttctttccagatcacgtggagctgtcctggtgggtcaacggcaaagaagtgcattctggggtctgcaccgacccccaggcttacaaggagagtaattactcatattgtctgtcaagccggctgagagtgtccgccacattctggcacaaccctaggaatcatttccgctgccaggtccagtttcacggcctgagtgaggaagataaatggccagaggggtcacctaagccagtgacacagaacatcagcgcagaagcctggggacgagcagactgtggcattactagcgcctcctatcatcagggcgtgctgagcgccactatcctgtacgagattctgctgggaaaggccaccctgtatgctgtgctggtctccggcctggtgctgatggccatggtcaagaaaaagaactcttga

(19) United States
(12) Patent Application Publication
(10) Pub. No.: US 2024/0009235 A1
(43) Pub. Date: Jan. 11, 2024

(54) T CELL RECEPTORS DIRECTED AGAINST BOB1 AND USES THEREOF

(71) Applicant: ACADEMISCH ZIEKENHUIS LEIDEN (H.O.D.N. LUMC), Leiden (NL)

(72) Inventors: Mirjam H.M. HEEMSKERK, Leiden (NL); J.H. Frederik FALKENBURG, Leiden (US)

(21) Appl. No.: 18/029,858

(22) PCT Filed: Sep. 22, 2021
(86) PCT No.: PCT/NL2021/050570

§ 371 Date: Mar. 31, 2023

(30) Foreign Application Priority Data
Oct. 2, 2020 (NL) 2026614

(51) Int. Cl. A61K 35/17; C12N 5/0783; C12N 15/86; C07K 14/47; A61K 39/00; A61P 35/00

(52) U.S. Cl. CPC A61K 35/17; C12N 5/0636; C12N 15/86; C07K 14/4705; A61K 39/4611; A61K 39/4632; A61P 35/00; A61K 2239/15

(57) ABSTRACT

Novel nucleic acid compositions, vector systems, modified cells and pharmaceutical compositions that encode or express T cell receptor components directed against Bob 1 are provided herein. These novel components may be used to enhance an immune response in a subject diagnosed with a hyperproliferative disease or condition. Associated methods for treating such subjects are therefore also provided herein.

Novel nucleic acid compositions, vector systems, modified cells and pharmaceutical compositions that encode or express T cell receptor components directed against Bob1 are provided herein. These novel components may be used to enhance an immune response in a subject diagnosed with a hyperproliferative disease or condition. Associated methods for treating such subjects are also provided herein.

### BACKGROUND

T cell activation is an important step in the protective immunity against pathogenic microorganisms (e.g., viruses, bacteria, and parasites), foreign proteins, and harmful chemicals in the environment, and also as immunity against cancer and other hyperproliferative diseases. T cells express receptors on their surfaces (i.e., T cell receptors) that recognize antigens presented on the surface of cells. During a normal immune response, binding of these antigens to the T cell receptor, in the context of MHC antigen presentation, initiates intracellular changes leading to T cell activation.

Adoptive T cell therapy has been used to treat hyperproliferative diseases, including tumors, by providing an antigen-specific immune response. One method involves the use of genetically modified T cells that express an antigen-specific protein having an extracellular domain that binds to an antigen.

### BRIEF SUMMARY OF THE DISCLOSURE

The intracellular transcription factor B cell Oct binding protein 1 (Bob1) encoded by gene POU2AF1 has previously been identified as a suitable target for TCR-based immunotherapies for B cell malignancies and multiple myeloma (see for example, WO2016/071758). Bob1 polypeptides are therefore useful targets for immunotherapy. TCR gene transfer approaches using Bob1-specific TCRs can bring novel treatment modalities for patients with B cell malignancies or multiple myeloma, among other diseases.

A T cell receptor specific to the Bob1 peptide LPHQPLATY (SEQ ID NO:5) when presented by MHC Class I HLA-B*35:01 has been identified herein, which recognizes primary B cell malignancies and multiple myeloma. Novel nucleic acid compositions, vector systems, modified cells and pharmaceutical compositions that encode or express T cell receptor components directed against Bob1 are therefore provided herein. These compositions and methods provide novel treatment modalities for MHC Class I HLA B*35:01 positive patients with B cell malignancies or multiple myeloma, among other diseases.

In one aspect, the invention provides a nucleic acid composition that encodes a Bob1 antigen-specific binding protein having a TCR α chain variable (Vα) domain and a TCR βchain variable (Vβ) domain, the composition comprising:
(a) a nucleic acid sequence that encodes a TCR Vα domain comprising a CDR3 amino acid sequence having at least 80% sequence identity to SEQ ID NO:12, or a functional fragment thereof; and
(b) a nucleic acid sequence that encodes a TCR Vβ domain comprising a CDR3 amino acid sequence having at least 80% sequence identity to SEQ ID NO: 21, or a functional fragment thereof.

Suitably, the composition may comprise:
(a) a nucleic acid sequence that encodes a TCR Vα domain comprising a CDR3 amino acid sequence having at least 90% sequence identity to SEQ ID NO:12, or a functional fragment thereof; and
(b) a nucleic acid sequence that encodes a TCR Vβ domain comprising a CDR3 amino acid sequence having at least 90% sequence identity to SEQ ID NO: 21, or a functional fragment thereof.

Suitably, the nucleic acid molecule may be an isolated nucleic acid molecule.

Suitably, the Bob1 antigen may comprise the amino acid sequence LPHQPLATY (SEQ ID NO:5).

Suitably, the encoded binding protein may be capable of specifically binding to a LPHQPLATY:HLA-B*35:01 complex. In other words, the CDR3 amino acid sequences of the composition may specifically bind to a peptide-MHC complex, wherein the peptide is a Bob1 epitope comprising the amino acid sequence of LPHQPLATY, and the MHC molecule is an MHC Class I HLA B*35:01 molecule.

Suitably, the nucleic acid sequence may be codon optimised for expression in a host cell. Optionally the host cell may be a human cell.

Suitably, (i) the CDR3 of the Vα domain may comprise or consist of the amino acid sequence of SEQ ID NO: 12, and (ii) the CDR3 of the Vβ domain may comprise or consist of the amino acid sequence of SEQ ID NO:21.

Suitably, (i) the CDR3 of the Vα domain may be encoded by a nucleic acid sequence comprising the sequence of SEQ ID NO: 13 or SEQ ID NO:14, or a derivative thereof; and/or (ii) the CDR3 of the Vβ domain may be encoded by a nucleic acid sequence comprising the sequence of SEQ ID NO: 22 or SEQ ID NO:23, or a derivative thereof.

Suitably, (i) the Vα domain may comprise an amino acid sequence having at least 80% sequence identity to, comprising, or consisting of, SEQ ID NO: 24, or a functional fragment thereof; and/or (ii) the Vβ domain may comprise an amino acid sequence having at least 80% sequence identity to, comprising, or consisting of, SEQ ID NO: 27, or a functional fragment thereof.

For example, (i) the Vα domain may comprise an amino acid sequence having at least 90% sequence identity to, comprising, or consisting of, SEQ ID NO: 24, or a functional fragment thereof; and/or (ii) the Vβ domain may comprise an amino acid sequence having at least 90% sequence identity to, comprising, or consisting of, SEQ ID NO: 27, or a functional fragment thereof. SEQ ID NO:24 represents the amino acid sequence of the VJ region of TCR 1C5.6 described herein whereas SEQ ID NO:27 represents the amino acid sequence of the VDJ region of TCR 1C5.6 described herein.

Suitably, (i) the Vα domain may be encoded by a nucleic acid sequence comprising the sequence of SEQ ID NO: 25 or SEQ ID NO: 26 or a derivative thereof; and/or (ii) the Vβ domain may be encoded by a nucleic acid sequence comprising the sequence of SEQ ID NO: 28 or SEQ ID NO:29, or a derivative thereof. SEQ ID NOs:25 and 26 represent nucleic acid sequences that encode the VJ region of TCR 1C5.6 described herein whereas SEQ ID NOs:28 and 29 represent nucleic acid sequences that encode the VDJ region of TCR 1C5.6 described herein.

Suitably, the nucleic acid composition may further comprise a TCR α chain constant domain and/or a TCR β chain constant domain.

Suitably, the constant domain may be a heterologous constant region.

Suitably, the constant domain may be derived from a murine TCR constant region.

Suitably, the Vα domain may comprise the amino acid sequence of SEQ ID NOs: 30 or 31. These sequences represent the amino acid sequence of the VJ region of TCR 1C5.6 and constant regions described herein.

Suitably, the Vα domain may be encoded by the nucleotide sequence of SEQ ID NOs: 32 or 33. These sequences represent the nucleic acid sequence of the VJ region of TCR 1C5.6 and the constant regions described herein.

Suitably, the Vβ domain may comprise the amino acid sequence of SEQ ID NOs: 34 or 35. These sequences represent the amino acid sequence of the VDJ region of TCR 1C5.6 and the constant regions described herein.

Suitably, the Vβ domain may be encoded by the nucleotide sequence of SEQ ID NOs: 36 or 37. These sequences represent the nucleic acid sequence of the VDJ region of TCR 1C5.6 and the constant regions described herein.

Suitably, the encoded binding protein may comprise a TCR, an antigen binding fragment of a TCR, or a chimeric antigen receptor (CAR).

Suitably, the antigen binding fragment of a TCR may be a single chain TCR (scTCR) or a chimeric TCR dimer in which the antigen binding fragment of the TCR is linked to an alternative transmembrane and intracellular signalling domain.

In another aspect, a vector system comprising a nucleic acid composition of the invention is provided.

Suitably, the vector may be a plasmid, a viral vector, or a cosmid. Optionally the vector may be selected from the group consisting of a retrovirus, lentivirus, adeno-associated virus, adenovirus, vaccinia virus, canary poxvirus, herpes virus, minicircle vector and synthetic DNA or RNA.

In another aspect, a modified (recombinant) cell comprising a nucleic acid composition of the invention or a vector system of the invention is provided.

Suitably, the modified cell may be selected from the group consisting of a CD8 T cell, a CD4 T cell, an NK cell, an NK-T cell, a gamma-delta T cell, an inducible pluripotent stem cell (iPSC), a hematopoietic stem cell, a progenitor cell, a T cell line and a NK-92 cell line.

Suitably, the modified cell may be a human cell.

Suitably, the modified cells may be autologous cells or allogeneic cells.

Suitably, the modified cells may be transfected or transduced in vitro, ex vivo, or in vivo.

In another aspect, a pharmaceutical composition comprising a nucleic acid composition of the invention, a vector system of the invention, or a modified cell of the invention, and a pharmaceutically acceptable excipient, adjuvant, diluent and/or carrier is provided.

The pharmaceutical composition described herein may be for use in inducing or enhancing an immune response in an HLA-B*35:01 positive human subject diagnosed with a hyperproliferative disease or condition.

Suitably, the subject diagnosed with a hyperproliferative disease or condition may have at least one tumor. Suitably, the size of the at least one tumor is reduced following administration of the pharmaceutical composition.

Suitably, the subject diagnosed with a hyperproliferative disease or condition may have been diagnosed with a B cell malignancy or multiple myeloma. Optionally, the B cell malignancy may be a B cell lymphoma or a B cell leukemia. Optionally, the B cell malignancy may be selected from the group consisting of mantle cell lymphoma, acute lymphoblastic leukemia, chronic lymphocytic leukemia, follicular lymphoma and large B cell lymphoma.

Suitably, the subject may have been diagnosed with acute lymphoblastic leukemia, chronic lymphocytic leukemia or multiple myeloma.

The pharmaceutical composition may additionally or alternatively be for use in stimulating a cell mediated immune response to a target cell population or tissue in an HLA-B*35:01 positive human subject.

Suitably, the target cells may express Bob1.

Suitably, the target cells may comprise a peptide-MHC cell surface complex, wherein the peptide is a Bob1 epitope comprising the amino acid sequence of LPHQPLATY, and the MHC molecule is an MHC Class I HLA B*35:01 molecule.

Suitably, the target cell may be a tumor cell.

Suitably, the target cell may be a B cell malignancy, a primary B cell malignancy, or a multiple myeloma cell. Suitably, the B cell malignancy may be a B cell lymphoma or a B cell leukemia, optionally wherein the B cell malignancy is selected from the group consisting of mantle cell lymphoma, acute lymphoblastic leukemia, chronic lymphocytic leukemia, follicular lymphoma and large B cell lymphoma.

Suitably, the number or concentration of target cells may be measured in a first sample obtained from the subject before administering the pharmaceutical composition, and the number or concentration of target cells may be measured in a second sample obtained from the subject after administration of the pharmaceutical composition. In this way, an increase or decrease of the number or concentration of target cells in the second sample compared to the number or concentration of target cells in the first sample may be determined. Suitably, the number or concentration of target cells in the subject may be reduced following administration of the pharmaceutical composition described herein.

The pharmaceutical composition may additionally or alternatively be for use in providing anti-tumor immunity to an HLA-B*35:01 positive human subject.

Suitably, the pharmaceutical composition may be used to provide immunity from a B cell malignancy, a primary B cell malignancy, or a multiple myeloma cell. Suitably, the B cell malignancy may be a B cell lymphoma or a B cell leukemia, optionally wherein the B cell malignancy is selected from the group consisting of mantle cell lymphoma, acute lymphoblastic leukemia, chronic lymphocytic leukemia, follicular lymphoma and large B cell lymphoma.

The pharmaceutical composition may additionally or alternatively be for use in treating an HLA-B*35:01 positive human subject having a disease or condition associated with an elevated level of Bob1.

Suitably, the elevated level of Bob1 may be associated with a tumor cell, such as a B cell malignancy, a primary B cell malignancy, or a multiple myeloma cell. Suitably, the B cell malignancy may be a B cell lymphoma or a B cell leukemia, optionally wherein the B cell malignancy is selected from the group consisting of mantle cell lymphoma, acute lymphoblastic leukemia, chronic lymphocytic leukemia, follicular lymphoma and large B cell lymphoma.

In another aspect, a method is provided for generating a binding protein that is capable of specifically binding to a peptide containing a Bob1 antigen and does not bind to a peptide that does not contain a Bob1 antigen, the method comprising contacting a nucleic acid composition of the invention with a cell under conditions in which the nucleic acid composition is incorporated and expressed by the cell.

Suitably, the binding protein may be capable of specifically binding to a peptide-MHC complex, wherein the peptide is a Bob1 antigen comprising the amino acid sequence of LPHQPLATY, and the MHC molecule is an MHC Class I HLA B*35:01 molecule.

Suitably, the nucleic acid composition may be contacted with the cell in vitro, ex vivo or in vivo. Suitably, the method may be ex vivo.

In another aspect, an isolated nucleic acid sequence is provided comprising or consisting of the nucleotide sequence of any one of SEQ ID NOs: 13, 14, 22, 23, 25, 26, 28, 29, 32, 33, 36 or 37.

In another aspect, an isolated nucleic acid sequence comprising or consisting of the nucleotide sequence of any one of SEQ ID NOs: 13, 14, 22, 23, 25, 26, 28, 29, 32, 33, 36 or 37 is provided for use in therapy.

In another aspect, a method of inducing or enhancing an immune response in an HLA-B*35:01 positive human subject diagnosed with a hyperproliferative disease or condition is provided, comprising administering an effective amount of a pharmaceutical composition of the invention to the subject.

In another aspect, a method for stimulating a cell mediated immune response to a target cell population or tissue in an HLA-B*35:01 positive human subject is provided, comprising administering an effective amount of a pharmaceutical composition of the invention to the subject.

In another aspect, a method for providing anti-tumor immunity to an HLA-B*35:01 positive human subject is provided, comprising administering to the subject an effective amount of a pharmaceutical composition of the invention.

In another aspect, a method for treating an HLA-B*35:01 positive human subject having a disease or condition associated with an elevated level of Bob1 is provided, comprising administering to the subject an effective amount of a pharmaceutical composition of the invention.

Suitably, the subject may have at least one tumor.

Suitably, the subject may have been diagnosed with a B cell malignancy or multiple myeloma, optionally wherein the B cell malignancy is a B cell lymphoma or a B cell leukemia. Optionally, the B cell malignancy may be selected from the group consisting of mantle cell lymphoma, acute lymphoblastic leukemia, chronic lymphocytic leukemia, follicular lymphoma and large B cell lymphoma.

In another aspect, the use of a pharmaceutical composition of the invention in the manufacture of a medicament for inducing or enhancing an immune response in an HLA-B*35:01 positive human subject diagnosed with a hyperproliferative disease or condition is provided.

In another aspect, the use of a pharmaceutical composition of the invention in the manufacture of a medicament for stimulating a cell mediated immune response to a target cell population or tissue in an HLA-B*35:01 positive human subject is provided.

In another aspect, the use of a pharmaceutical composition of the invention in the manufacture of a medicament for providing anti-tumor immunity to an HLA-B*35:01 positive human subject is provided.

In another aspect, the use of a pharmaceutical composition of the invention in the manufacture of a medicament for treating an HLA-B*35:01 positive human subject having a disease or condition associated with an elevated level of Bob1 is provided.

Suitably, the subject may have at least one tumor.

Suitably, the subject may have been diagnosed with a B cell malignancy or multiple myeloma, optionally wherein the B cell malignancy is a B cell lymphoma or a B cell leukemia. Optionally, the B cell malignancy may be selected from the group consisting of mantle cell lymphoma, acute lymphoblastic leukemia, chronic lymphocytic leukemia, follicular lymphoma and large B cell lymphoma.

Throughout the description and claims of this specification, the words “comprise”, and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 shows a gene expression profile of the POU2AF1 gene encoding the Bob1 protein. Gene expression was previously determined by illumina HT12.0 microarray. POU2AF1 expression (Mean Fluorescent Intensity; MFI) per cell type, individual samples and average (mean) gene expression is shown. Expression in patient derived B cell malignancies or B cell malignancy cell lines (left panel), healthy peripheral blood B cells (CD19^{pos}) or B cell containing subsets (middle panel), healthy hematopoietic and non-hematopoietic cell subsets (right panel).

FIG. 2 shows matching tandem mass spectra of eluted (top) and synthetic (bottom) peptide p236 LPHQPLATY derived from Bob1 presented in HLA-B*35:01 (HLA-B*35).

FIG. 3 shows potency screening of T cell clone 1C5.6 and clone 4H5.6. (A) T cell clones 1C5.6 and 4H5.6 were stimulated with a 1:1 mixture of HLA-B8 and HLA-B35 Td K562 cells, loaded with combinatorial peptide mixes (100 nM) to identify peptide specificity (upper part) and K562 cells Td with target gene+HLA (bottom part) to determine recognition of endogenously processed and presented peptide (bottom part). IFN-γ production was measured by ELISA after overnight (O/N) co-culture. (B) T cell clone 1C5.6 and 4H5.6 stained with PE-labeled Bob1 tetramers p233 (APA) and p236 (LPH) showed specific binding to Bob1 tetramer p236 (LPH) (right peak). (C) IFN-γ production by T cell clones 1C5.6 and 4H5.6 after O/N stimulation HLA-B35 Td K562 cells loaded with decreasing concentrations of target peptide p236 (LPH). (D) IFN-γ production after O/N stimulation with different acute lymphoblastic leukemia (ALL) cell lines, multiple myeloma (MM) cell lines and Bob1 negative K562 cells. Target cells were positive (+), negative (−) or transduced (Td) with HLA-B35.

FIG. 4 shows safety screening of the most potent Bob1 specific HLA-B*35:01 restricted T cell clone 1C5.6. (A) IFN-γ production by T cell clone 1C5.6 after O/N co-culture with an EBV-LCL panel expressing HLA class I alleles with a frequency >1% in the Caucasian population but not HLA-B*35:01. HLA-B*35:01 and POU2AF1 gene (Bob1) Td K562 cells were used as positive control for T cell function. (B) IFN-γ production after O/N co-culture with HLA-B35 Td tumor cell lines of multiple non-B cell origins and positive control K562 cells.

FIG. 5 shows CD8 T cell functionality after retroviral gene transfer of TCR 1C5.6. (A) CD8 T cells unTd (left panel), Td with negative control CMV (pp65-HLA-A2) TCR (middle panel) or Bob1 HLA-B35 TCR 1C5.6 (right panel) both containing murine TCR constant beta domains (mTCRcβ), enriched for mTCRcβ expression day 10 after activation. T cells were stained with tetramer-PE mix and mTCRcβ-APC, analyzed by FACS. (B) IFN-γ production after O/N co-culture with HLA-B35 Td K562 cells as negative control and HLA-B35 and POU2AF1 gene (Bob1) Td K562 cells as positive control. An allo HLA-B35 T cell clone was included as control for target HLA expression.

FIG. 6 shows antigen dependent killing of B cell malignancies by TCR 1C5.6 Td CD8 T cells. (A) Killing by CD8 T cells Td with TCRs 1C5.6 (circles), CMV (pp65-HLA-A2) TCR Td CD8 T cells (triangles) as negative control and allo HLA-B35 T cell clone (inverted triangles) as positive controls. Target cells were primary B cell malignancies (top row), HLA-B*35:01 positive or negative B cell malignancy cell lines, HLA-B35 negative cell lines were Td with HLA-B*35:01 or irrelevant HLA-A24 (middle row), antigen negative HLA-B*35:01 positive fibroblasts and keratinocytes pretreated for 48 hours with 100 IU/ml IFN-γ and K562 cells (bottom row). Killing was measured by 51CR release assay after 6 hour co-culture in different E:T ratios. Values and error bars represent mean and standard deviations of technical triplicates. (B) IFN-γ production after O/N co-culture of T cells and target cells used in (A) and peptide (p236, LPHQPLATY) loaded HLA-B35 Td K562 cells as positive control. Abbreviations: ALL, acute lymphoblastic leukemia; CLL, chronic lymphocytic leukemia; MCL, mantle cell lymphoma; MM, multiple myeloma; DLBCL, diffuse large B cell lymphoma.

FIG. 7. In vivo antitumor efficacy of BOB1 HLA-B35 restricted TCR transduced CD8 T cells. NSG mice engrafted with 2×106 U266 multiple myeloma cells transduced with luciferase and HLA-B35, were i.v. injected with 5×106 TCR transduced CD8 T cells after 21 days. T cells were transduced with BOB1 HLA-B35 restricted TCR 1C5.6 (n=4) or control CMV (pp65-NLV-HLA-A2) TCR (n=3) and enriched for mTCR expression by MACS. Tumor outgrowth was frequently tracked by bioluminescence imaging. (A) Mean and standard deviations of tumor outgrowth over time on the ventral side of CMV TCR treated control mice (dashed line) and BOB1 HLA-B35 TCR (solid line) treated mice. (B) Tumor outgrowth for individual CMV TCR (left) or BOB1 HLA-B35 TCR (right) treated mice measured on day 20, 27, 34 and 48 after tumor cell injection.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

Adoptive T cell therapy has been used to treat hyperproliferative diseases, including tumors, by providing an antigen-specific immune response. One method involves the use of genetically modified T cells that express an antigen-specific protein having an extracellular domain that binds to an antigen. Recombinant T cell receptors have been used to provide specificity to T cells. In other methods, heterologous T cell receptors, specific for a particular antigen, have been expressed in T cells to provide an antigen-specific immune response. Methods of adoptive T cell therapy are well known in the art, see for example WO2016/071758.

Methods of adoptive T cell therapy have often targeted extracellular antigens. For example, CD19, an extracellular antigen on the surface of B cell malignancies, has been a target for T cell therapy. However, using a CD19-specific antigen receptor-transduced T cell may not be as effective when the B cell malignancy loses expression of the CD19 antigen. Thus, where, for example, T cells are engineered to recognize CD20, or CD19, the loss of CD20 and CD19 expression or absence of these molecules on other malignancies such as multiple myeloma restricts their application.

An intracellular transcription factor Bob1, encoded by gene POU2AF1, has previously been found to be a suitable target for immunotherapy. Bob1 is highly expressed in CD19⁺ B cells, acute lymphoblastic leukemia (ALL), chronic lymphocytic leukemia (CLL), mantle cell lymphoma (MCL), follicular lymphoma, large B cell lymphoma, and multiple myeloma (MM) and is absent in the non-B lineages including CD34⁺ hematopoietic progenitor cells (HPCs), T cells, fibroblasts, keratinocytes and gastrointestinal tract.

Bob1 is localized intracellularly, but HLA-presented Bob1-derived polypeptides are accessible on the cell surface to T cell receptors (TCRs) and can thus be recognized by T cells. From the HLA-presented ligandome (Mol Cell Proteomics, 2013; 12:1829) naturally processed Bob1-derived polypeptides have been identified that are displayed in HLA-A*02:01 (HLA-A2), HLA-B*07:02 (HLA-B7), and HLA-B*35:01 (Tables 2 and 3). Since auto-reactivity toward self-antigens such as Bob1 is prevented by depleting high-avidity T cells recognizing self-antigens in self-HLA, the immunogenicity of these polypeptides presented in allogeneic HLA was exploited.

To isolate potent T cell clones recognizing target peptides derived from selected B cell specific genes, including Bob1, a mixture of 20 different pHLA-tetramers were incubated with peripheral blood mononuclear cells (PBMCs) from healthy donors negative for the target HLA alleles. The pHLA-tetramers were composed of 20 different B cell specific peptides binding in either HLA-A*01:01, A*24:02, B*08:01, or B*35:01. pHLA-tetramer bound cells were enriched by MACS and pHLA-tetramer CD8⁺ T cells were single cell sorted using FACS. Buffy coats consisting of 1-3×10⁹ PBMCs from 13 donors were used, in total 12336 T cells were single cell sorted. On average 59% (14%-83%) of T cell clones expanded.

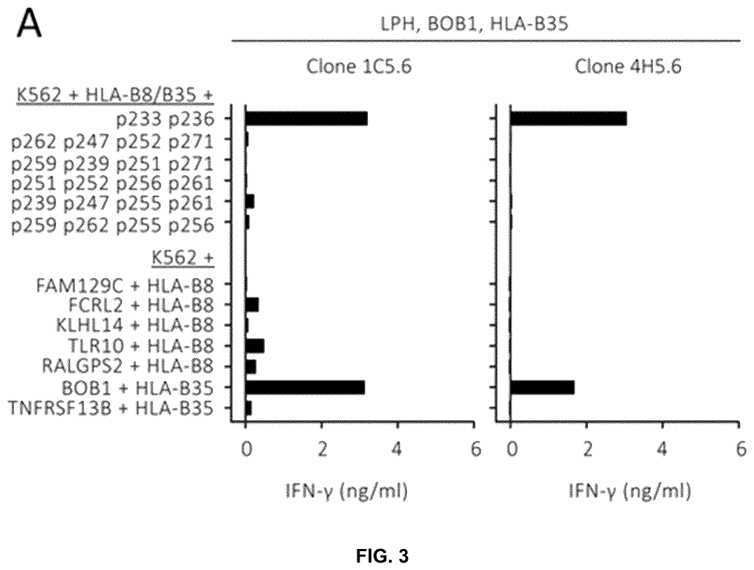

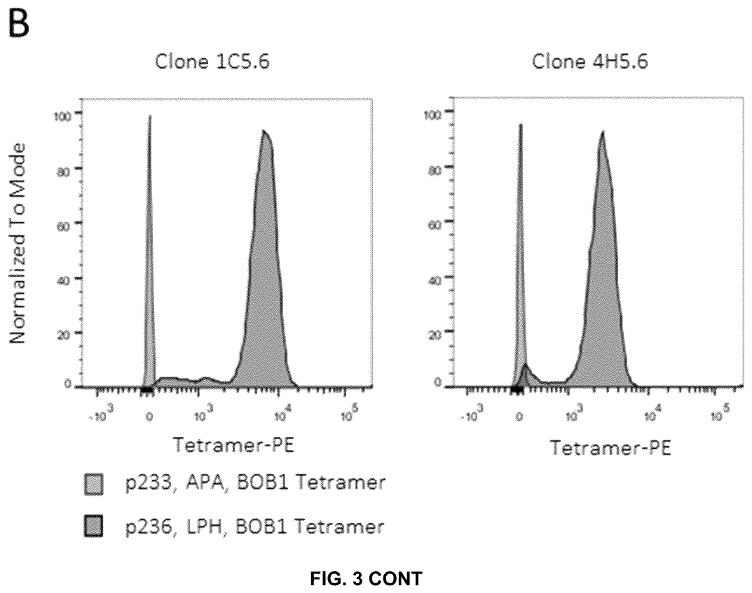

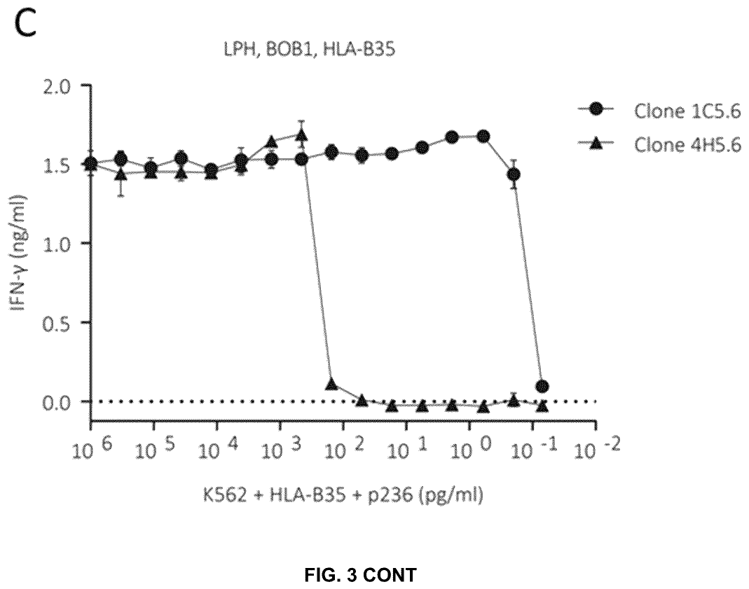

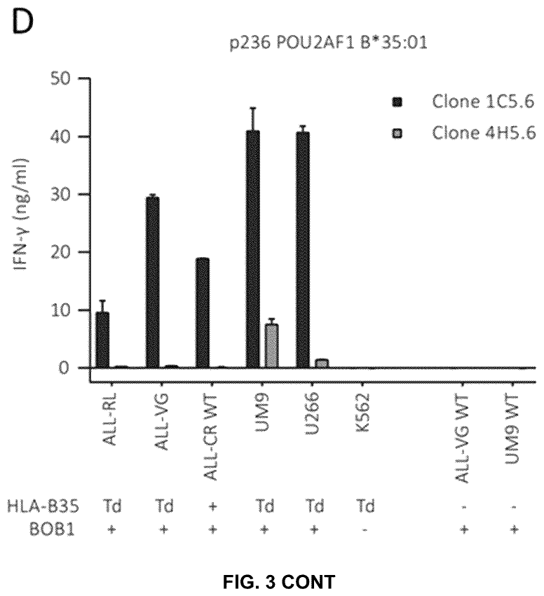

To select peptide specific T cell clones, the T cell clones were co-incubated with K562 cells transduced (Td) with target HLA alleles either alone or loaded with a mixture of target peptides, and after overnight stimulation the supernatant was harvested to measure cytokine production. This revealed lack of functionality, measured by IFN-γ production, in 34-98% of expanded T cell clones. Additionally, target HLA restricted K562 recognition irrespective of peptide addition was frequently observed, these clones were discarded to prevent off-target toxicity. A total of 46 T cell clones specifically recognized peptide loaded cells but not the unloaded cells and were selected for further functional analysis. From these 46 T cell clones only 2 clones, clone 1C5.6 and clone 4H5.6, derived from the buffy coats of 2 different donors, were specific for the Bob1 peptide 236 with the amino acid sequence LPHQPLATY, recognized in the context of HLA-B*35:01 (FIG. 3A). To identify the T cell clone with the highest affinity, the two T cell clones were compared for peptide-sensitivity by testing the recognition of stimulator cells loaded with titrated amounts of Bob1-derived HLA-B*35:01 binding peptide. Clone 1C5.6 demonstrated to be the T cell clone with the highest affinity, since this T cell clone was still efficiently activated with a more then 100 fold lower concentration of Bob1 peptide compared to clone 4H5.6 (FIG. 3B). In addition, clone 1C5.6 efficiently recognized all the Bob1 positive HLA-B*35:01 positive B-cell malignant cell lines, in contrast to clone 4H5.6 which only recognized 2 out of 5 B-cell malignant cell lines (FIG. 3D). Therefore, the TCR of clone 1C5.6 was selected as the most potent Bob1 specific HLA-B*35:01 restricted TCR for further analyses.

The TCR components of clone 1C5.6 form the basis of the invention and are described in more detail herein. These sequences are shown herein to bind to the HLA-B*35:01 restricted BOB1 peptide of SEQ ID NO:5 with high specificity. They also recognize the HLA-B*35:01 restricted BOB1 peptide of SEQ ID NO:5 with high affinity, since 1C5.6 TCR was efficiently activated with a more than 100 fold lower concentration of Bob1 peptide compared to TCR 4H5.6. Furthermore, they are safe, as no cross reactivity to any HLA-I alleles with a frequency >1% in the Caucasian population was observed, and no reactivity against HLA-B*35:01 positive cell lines of multiple non-B cell origins, was observed.

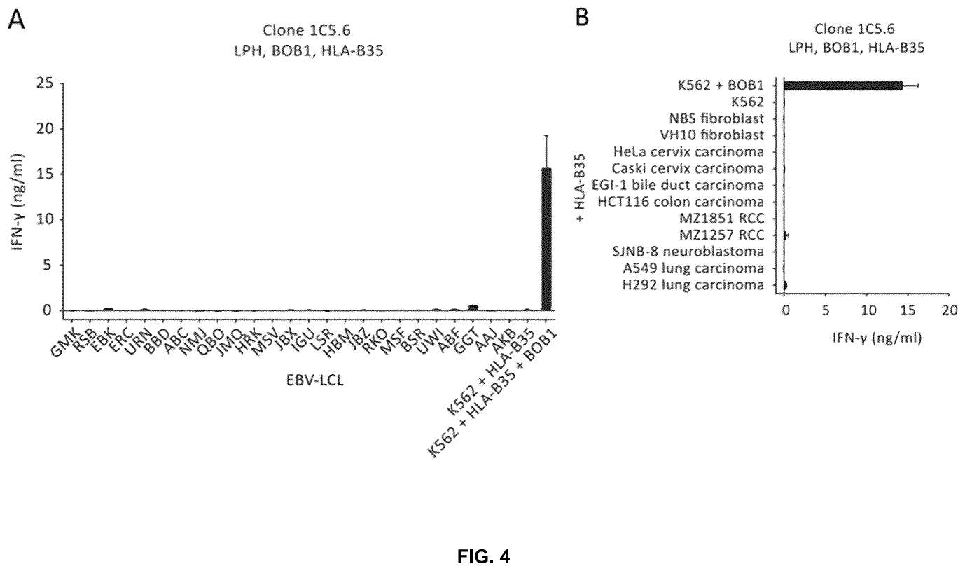

The TCR components described herein may therefore be described as TCR components that bind to the HLA-B*35:01 restricted BOB1 peptide of SEQ ID NO:5 with high specificity. In addition, or alternatively, they may be described as TCR components that recognize the HLA-B*35:01 restricted BOB1 peptide of SEQ ID NO:5 with high affinity. Additionally, or alternatively, they may be described as TCR components that have no cross reactivity to any HLA-I alleles with a frequency >1% in the Caucasian population (as per Table 3), and no reactivity against H LA-B*35:01 positive cell lines of multiple non-B cell origins (FIG. 4).

### Nucleic Acid Compositions that Encode Binding Protein Components

The invention provides an isolated nucleic acid composition that encodes a binding protein comprising T cell receptor (TCR) components that specifically bind a Bob1 antigen. The encoded binding protein is therefore capable of specifically binding to a peptide containing a Bob1 antigen (specifically comprising the sequence LPHQPLATY (SEQ ID NO:5)) and does not bind to a peptide that does not contain a Bob1 antigen (specifically comprising the sequence LPHQPLATY (SEQ ID NO:5)).

The nucleic acid composition comprises (a) a nucleic acid sequence that encodes a TCR Vα domain with the specified features described herein and (b) a nucleic acid sequence that encodes a TCR Vβ domain with the specified features described herein. The encoded TCR components form a Bob1 antigen-specific binding protein.

The nucleic acid sequences of (a) and (b) above may be distinct nucleic acid sequences within the nucleic acid composition. The TCR components of the binding protein may therefore be encoded by two (or more) nucleic acid sequences (with distinct nucleotide sequences) which, together, encode all of the TCR components of the binding protein. In other words, some of the TCR components may be encoded by one nucleic acid sequence in the nucleic acid composition, and others may be encoded by another (distinct) nucleic acid sequence within the nucleic acid composition.

Alternatively, the nucleic acid sequences of (a) and (b) may be part of a single nucleic acid sequence. The TCR components of the binding protein may therefore all be encoded by a single nucleic acid sequence (for example with a single open reading frame, or with multiple (e.g. 2 or more, three or more etc.) open reading frames).

Nucleic acid sequences described herein may form part of a larger nucleic acid sequence that encodes a larger component part of a functioning binding protein. For example, a nucleic acid sequence that encodes a TCR Vα domain with the specified features described herein may be part of a larger nucleic acid sequence that encodes a functional TCR α chain (including the constant domain). As another example, a nucleic acid sequence that encodes a TCR Vβ domain with the specified features described herein may be part of a larger nucleic acid sequence that encodes a functional TCR β chain (including the constant domain). As a further example, both nucleic acid sequences (a) and (b) above may be part of a larger nucleic acid sequence that encodes a combination of a functional TCR α chain (including the constant domain) and a functional TCR β chain (including the constant domain), optionally wherein the sequence encoding the functional TCR α chain is separated from the sequence encoding the functional TCR β chain by a linker sequence that enables coordinate expression of two proteins or polypeptides in the same nucleic acid sequence. More details on this are provided below.

The nucleic acid sequences described herein may alternatively encode a small component of a T cell receptor e.g. a TCR Vα domain, or a TCR Vβ domain, only. The nucleic acid sequences may be considered as “building blocks” that provide essential components for peptide binding specificity. The nucleic acid sequences described herein may be incorporated into a distinct nucleic acid sequence (e.g. a vector) that encodes the other elements of a functional binding protein such as a TCR, such that when the nucleic acid sequence described herein is incorporated, a new nucleic acid sequence is generated that encodes e.g. a TCR α chain and/or a TCR β chain that specifically binds to a Bob1 antigen. The nucleic acid sequences described herein therefore have utility as essential components that confer binding specificity for a Bob1 antigen, and thus can be used to generate a larger nucleic acid sequence encoding a binding protein with the required antigen binding activity and specificity.

The nucleic acid sequences described herein may be codon optimised for expression in a host cell, for example they may be codon optimised for expression in a human cell, such as a cell of the immune system, a inducible pluripotent stem cell (iPSC), a hematopoietic stem cell, a T cell, a primary T cell, a T cell line, a NK cell, or a natural killer T cell (Scholten et al, Clin. Immunol. 119: 135, 2006). The T cell can be a CD4+ or a CD8+ T cell. Codon optimisation is a well-known method in the art for maximizing expression of a nucleic acid sequence in a particular host cell. As described in the examples section below, one or more cysteine residues may also be introduced into the encoded TCR alpha and beta chain components (e.g. to reduce the risk of mispairing with endogenous TCR chains).

In one example, the nucleic acid sequences described herein are codon optimised for expression in a suitable host cell, and/or are modified to introduce codons encoding one or more cysteine amino acids (e.g. into the constant domain of the encoded TCR alpha chain and/or the encoded TCR beta chain) to reduce the risk of mispairing with endogenous TCR chains.

In certain examples, a TCR constant domain is modified to enhance pairing of desired TCR chains. For example, enhanced pairing between a heterologous TCR α chain and a heterologous TCR β chain due to a modification may result in the preferential assembly of a TCR comprising two heterologous chains over an undesired mispairing of a heterologous TCR chain with an endogenous TCR chain (see, e.g., Govers et al, Trends Mol. Med. 16(2):11 (2010)). Exemplary modifications to enhance pairing of heterologous TCR chains include the introduction of complementary cysteine residues in each of the heterologous TCR α chain and β chain. In some examples, a polynucleotide encoding a heterologous TCR α chain encodes a cysteine at amino acid position 48 (corresponding to the constant region of the full-length, mature human TCR α chain sequence) and a polynucleotide encoding a heterologous TCR β chain encodes a cysteine at amino acid position 57 (corresponding to the constant region of the full-length mature human TCR β chain sequence).

A binding protein that is encoded by the nucleic acid compositions described herein is specific for a Bob1 antigen and comprises Bob1 antigen specific-TCR components. However, the encoded binding protein is not limited to being a TCR. Other appropriate binding proteins that comprise the specified Bob1 antigen specific-TCR components are also encompassed. For example, the encoded binding protein may comprise a TCR, an antigen binding fragment of a TCR, or a chimeric antigen receptor (CAR). TCRs, antigen binding fragments thereof and CARs are well defined in the art. A non-limiting example of an antigen binding fragment of a TCR is a single chain TCR (scTCR) or a chimeric dimer composed of the antigen binding fragments of the TCR α and TCR β chain linked to transmembrane and intracellular domains of a dimeric complex so that the complex is a chimeric dimer TCR (cdTCR).

In certain examples, an antigen-binding fragment of a TCR comprises a single chain TCR (scTCR), which comprises both the TCR Vα and TCR Vβ domains, but only a single TCR constant domain. In other examples, an antigen-binding fragment of a TCR comprises a chimeric TCR dimer in which the antigen binding fragment is linked to an alternative transmembrane and intracellular signalling domain (where the alternative transmembrane and intracellular signalling domain are not naturally found in TCRs). In further examples, an antigen-binding fragment of a TCR or a chimeric antigen receptor is chimeric (e.g., comprises amino acid residues or motifs from more than one donor or species), humanized (e.g., comprises residues from a non-human organism that are altered or substituted so as to reduce the risk of immunogenicity in a human), or human.

“Chimeric antigen receptor” (CAR) refers to a fusion protein that is engineered to contain two or more naturally-occurring amino acid sequences linked together in a way that does not occur naturally or does not occur naturally in a host cell, which fusion protein can function as a receptor when present on a surface of a cell. CARs described herein include an extracellular portion comprising an antigen binding domain (i.e., obtained or derived from an immunoglobulin or immunoglobulin-like molecule, such as an scFv derived from an antibody or TCR specific for a cancer antigen, or an antigen binding domain derived or obtained from a killer immunoreceptor from an NK cell) linked to a transmembrane domain and one or more intracellular signalling domains (optionally containing co-stimulatory domain(s)) (see, e.g., Sadelain et al, Cancer Discov., 3(4):388 (2013); see also Harris and Kranz, Trends Pharmacol. Sci., 37(3):220 (2016), and Stone et al, Cancer Immunol. Immunother., 63(11): 1163 (2014)).

Methods for producing engineered TCRs are described in, for example, Bowerman et al, Mol. Immunol, 5(15):3000 (2009). Methods for making CARs are well known in the art and are described, for example, in U.S. Pat. Nos. 6,410,319; 7,446,191; U.S. Patent Publication No. 2010/065818; U.S. Pat. No. 8,822,647; PCT Publication No. WO 2014/031687; U.S. Pat. No. 7,514,537; and Brentjens et al, 2007, Clin. Cancer Res. 73:5426.

The binding proteins described herein may also be expressed as part of a transgene construct that encodes additional accessory proteins, such as a safety switch protein, a tag, a selection marker, a CD8 co-receptor β-chain, α-chain or both, or any combination thereof.

A T cell receptor (TCR) is a molecule found on the surface of T cells (T lymphocytes) that is responsible for recognising a peptide that is bound to (presented by) a major histocompatibility complex (MHC) molecule on a target cell. The invention is directed to nucleic acid compositions that encode binding proteins comprising TCR components that interact with a particular peptide in the context of the appropriate serotype of MHC, i.e. a Bob1 antigen in the context of HLA-B*35:01 (in other words, the encoded binding protein is capable of specifically binding to a Bob1 antigen:HLA-B*35:01 complex). HLA-B*35:01 is a globally common human leukocyte antigen serotype within the HLA-B serotype group. Peptides that are presented by HLA-B*35:01 to TCRs are described as being “H LA-B*35:01 restricted”.

The Bob1 antigen that is specifically bound by the binding proteins described herein comprises the amino acid sequence shown in SEQ ID NO:5. The antigen may be an antigenic fragment (i.e. a portion) of the sequence shown in SEQ ID NO:5, it may consist of the sequence of SEQ ID NO:5 or it may comprise (i.e. include within a longer sequence) the sequence of SEQ ID NO:5. The Bob1 antigen is capable of being presented by HLA-B*35:01. The encoded binding protein may therefore be capable of specifically binding to a Bob1 antigen:HLA-B*35:01 complex, wherein the Bob1 antigen is an antigenic fragment of the sequence shown in SEQ ID NO:5, or wherein the Bob1 antigen comprises or consists of the amino acid sequence shown in SEQ ID NO: 5.

The TCR is composed of two different polypeptide chains. In humans, 95% of TCRs consist of an alpha (α) chain and a beta (β) chain (encoded by TRA and TRB respectively). When the TCR engages with peptide in the context of HLA (e.g. in the context of HLA-B*35:01), the T cell is activated through signal transduction.

The alpha and beta chains of the TCR are highly variable in sequence. Each chain is composed of two extracellular domains, a variable domain (V) and a constant domain (C). The constant domain is proximal to the T cell membrane followed by a transmembrane region and a short cytoplasmic tail while the variable domain binds to the peptide/HLA-A complex.

The variable domain of each chain has three hypervariable regions (also called complementarity determining regions (CDRs)). Accordingly, the TCR alpha variable domain (referred to herein as a TCR Vα domain, TCR V alpha domain, Vα domain or V alpha domain, alpha variable domain etc) comprises a CDR1, a CDR2 and CDR3 region. Similarly, the TCR beta variable domain (referred to herein as a TCR Vβ domain, TCR V beta domain, Vβ domain or V beta domain, beta variable domain etc) also comprises a (different) CDR1, CDR2, and CDR3 region. In each of the alpha and beta variable domains it is CDR3 that is mainly responsible for recognizing the peptide being presented by the HLA molecules.

As will be clear to a person of skill in the art, the phrase “TCR α chain variable domain” refers to the variable (V) domain (extracellular domain) of a TCR alpha chain, and thus includes three hypervariable regions (CDR1, CDR2 and the specified CDR3), as well as the intervening sequences, but does not include the constant (C) domain of the alpha chain, which does not form part of the variable domain.

As will be clear to a person of skill in the art, the phrase “TCR β chain variable domain” refers to the variable (V) domain (extracellular domain) of a TCR beta chain, and thus includes three hypervariable regions (CDR1, CDR2 and the specified CDR3), as well as the intervening sequences, but does not include the constant (C) domain of the beta chain, which does not form part of the variable domain.

An isolated nucleic acid composition that encodes a Bob1 antigen-specific binding protein having a TCR α chain variable (Vα) domain and a TCR β chain variable (Vβ) domain is provided herein, the composition comprising:
(a) a nucleic acid sequence that encodes a TCR Vα domain comprising a CDR3 amino acid sequence having at least 80% sequence identity to SEQ ID NO:12, or a functional fragment thereof; and
(b) a nucleic acid sequence that encodes a TCR Vβ domain comprising a CDR3 amino acid sequence having at least 80% sequence identity to SEQ ID NO: 21, or a functional fragment thereof.

Any of the permutations described below for (a) may be combined with the permutations described below for (b) (e.g. to form an appropriate nucleic acid composition that encodes a Bob1 antigen-specific binding protein having a TCR α chain variable (Vα) domain and a TCR β chain variable (Vβ) domain).

### Components of the TCR α Chain Variable (Vα) Domain

The isolated nucleic acid composition described herein encodes a Bob1 antigen-specific binding protein. The Bob1 antigen-specific binding protein comprises a TCR Vα domain comprising a CDR3 amino acid sequence having at least 80% sequence identity to SEQ ID NO: 12.

An example of an appropriate TCR Vα domain CDR3 amino acid sequence that confers specific binding to a Bob1 antigen is shown in SEQ ID NO:12. As would be clear to a person of skill in the art, variants of the amino acid sequence shown in SEQ ID NO:12 may also be functional (i.e. retain their ability to confer specific binding to a Bob1 antigen (e.g. the peptide shown in SEQ ID NO:5) when the CDR3 is part of TCR Vα domain). Such functional variants are therefore encompassed herein.

For example, appropriate (functional) Vα domain CDR3 amino acid sequences may have at least 80% sequence identity to SEQ ID NO: 12, i.e. they may have at least 80%, at least 83%, at least 85%, at least 90%, at least 91%, at least 92%, at least 95%, at least 96%, at least 97%, at least 98%, at least 99%, or 100% sequence identity to SEQ ID NO: 12. Suitably, percent identity is calculated as the percentage of identity to the entire length of the reference sequence (e.g. SEQ ID NO:12). In other words, appropriate (functional) Vα domain CDR3 amino acid sequences may vary from the sequence shown in SEQ ID NO:12 by one or several (e.g. two etc) amino acids.

As stated above, functional variants of SEQ ID NO:12 retain their ability to confer specific binding to a Bob1 antigen (e.g. the peptide shown in SEQ ID NO:5) when the CDR3 is part of TCR Vα domain.

Functional variants may be naturally occurring, synthetic, or synthetically improved functional variants of SEQ ID NO:12. The term “variant” also encompasses homologues and fragments. Functional variants will typically contain only conservative substitutions of one, two or more amino acids of SEQ ID NO:12, or substitution, deletion or insertion of non-critical amino acids in non-critical regions of the CDR3.

Non-functional variants are amino acid sequence variants of SEQ ID NO: 12 that do not specifically bind to a Bob1 antigen (e.g. the peptide shown in SEQ ID NO:5). Non-functional variants will typically contain a non-conservative substitution, a deletion, or insertion or premature truncation of the amino acid sequence of SEQ ID NO:12 or a substitution, insertion or deletion in critical amino acids or critical regions. Methods for identifying functional and non-functional variants are well known to a person of ordinary skill in the art.

In one example, the CDR3 of the Vα domain comprises or consists of the amino acid sequence of SEQ ID NO: 12. In examples where the TCR Vα domain CDR3 has the amino acid sequence of SEQ ID NO:12, the CDR3 may be encoded by the nucleic acid sequence of SEQ ID NO:13 or SEQ ID NO:14, or a genetically degenerate sequence thereof (i.e. other nucleic acid sequences that encode the same protein as a result of the degeneracy of the genetic code). It is noted that SEQ ID NO:14 is the codon optimised version of the nucleic acid sequence for CDR3 of clone 1C5.6 (the non-optimised sequence being SEQ ID NO:13).

The phrase “genetically degenerate sequence thereof” is used interchangeably with “derivative thereof” herein.

The encoded TCR Vα domain may comprise, in addition to the specified CDR3, a CDR1 comprising an amino acid sequence of SEQ ID NO: 6, or a functional variant thereof (i.e. wherein the variant retains the ability to specifically bind to the Bob1 antigen (e.g. the peptide shown in SEQ ID NO:5)). Such functional variants may be naturally occurring, synthetic, or synthetically improved functional variants of SEQ ID NO:6. The term “variant” also encompasses homologues and fragments. Functional variants will typically contain only conservative substitutions of one or more amino acids of SEQ ID NO:6, or substitution, deletion or insertion of non-critical amino acids in non-critical regions of the protein.

Non-functional variants are amino acid sequence variants of SEQ ID NO: 6 that do not specifically bind to the Bob1 antigen (e.g. the peptide shown in SEQ ID NO:5). Non-functional variants will typically contain a non-conservative substitution, a deletion, or insertion or premature truncation of the amino acid sequence of SEQ ID NO:6 or a substitution, insertion or deletion in critical amino acids or critical regions. Methods for identifying functional and non-functional variants are well known to a person of ordinary skill in the art.

For example, appropriate functional Vα domain CDR1 amino acid sequences may have at least 80% sequence identity to SEQ ID NO: 6, i.e. it may have at least 80%, at least 90%, at least 91%, at least 92%, at least 93%, at least 94%, at least 95%, at least 96%, at least 97%, at least 98%, at least 99%, or 100% sequence identity to SEQ ID NO: 6. Suitably, percent identity is calculated as the percentage of identity to the entire length of the reference sequence (e.g. SEQ ID NO:6). In other words, appropriate functional Vα domain CDR1 amino acid sequences may vary from the sequence shown in SEQ ID NO: 6 by one or several amino acids. As stated previously, the variant may comprise an amino acid substitution such as a conservative amino acid substitution compared to the sequence shown in SEQ ID NO:6). As stated above, functional variants of SEQ ID NO: 6 retain the ability to specifically bind to the Bob1 antigen (e.g. the peptide shown in SEQ ID NO:5) when the CDR1 is part of TCR Vα domain).

In one example, the CDR1 of the Vα domain comprises or consists of the amino acid sequence of SEQ ID NO:6. In examples where the TCR Vα domain CDR1 has the amino acid sequence of SEQ ID NO:6, the CDR1 may be encoded by the nucleic acid sequence of SEQ ID NO:7 or SEQ ID NO:8, or a genetically degenerate sequence thereof (i.e. other nucleic acid sequences that encode the same protein as a result of the degeneracy of the genetic code). It is noted that SEQ ID NO:8 is the codon optimised version of the nucleic acid sequence for CDR1 of clone 1C5.6 (the non-optimised sequence being SEQ ID NO:7).

The encoded TCR Vα domain may also comprise, in addition to the specified CDR3 (and optionally the specified CDR1 above), a CDR2 comprising an amino acid sequence of SEQ ID NO:9, or a functional variant thereof (i.e. wherein the variant retains the ability to specifically bind to HLA-B*35:01). Such functional variants may be naturally occurring, synthetic, or synthetically improved functional variants of SEQ ID NO:9. The term “variant” also encompasses homologues and fragments. Functional variants will typically contain only conservative substitutions of one or more amino acids of SEQ ID NO:9, or substitution, deletion or insertion of non-critical amino acids in non-critical regions of the protein.

Non-functional variants are amino acid sequence variants of SEQ ID NO: 9 that do not specifically bind to HLA-B*35:01. Non-functional variants will typically contain a non-conservative substitution, a deletion, or insertion or premature truncation of the amino acid sequence of SEQ ID NO: 9 or a substitution, insertion or deletion in critical amino acids or critical regions. Methods for identifying functional and non-functional variants are well known to a person of ordinary skill in the art.

For example, appropriate functional Vα domain CDR2 amino acid sequences may have at least 80% sequence identity to SEQ ID NO: 9, i.e. it may have at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 9. Suitably, percent identity is calculated as the percentage of identity to the entire length of the reference sequence (e.g. SEQ ID NO:9). In other words, appropriate (functional) Vα domain CDR2 amino acid sequences may vary from the sequence shown in SEQ ID NO:9 by one or several amino acids. As stated previously, the variant may comprise an amino acid substitution such as a conservative amino acid substitution compared to the sequence shown in SEQ ID NO:9).

As stated above, a functional variant of SEQ ID NO: 9 retains the ability to specifically bind to HLA-B*35:01.

In one example, the CDR2 of the Vα domain comprises or consists of the amino acid sequence of SEQ ID NO: 9. In examples where the TCR Vα domain CDR2 has the amino acid sequence of SEQ ID NO:9, the CDR2 may be encoded by the nucleic acid sequence of SEQ ID NO:10 or SEQ ID NO:11, or a genetically degenerate sequence thereof (i.e. other nucleic acid sequences that encode the same protein as a result of the degeneracy of the genetic code). It is noted that SEQ ID NO:11 is the codon optimised version of the nucleic acid sequence for CDR2 of clone 1C5.6 (the non-optimised sequence being SEQ ID NO:10).

The encoded TCR Vα domain may therefore comprise the CDRs mentioned in detail above (by SEQ ID specifically i.e. SEQ ID NO:12, SEQ ID NO: 6 and SEQ ID NO: 9, or functional variants thereof), with appropriate intervening sequences between the CDRs.

The encoded TCR Vα domain may comprise an amino acid sequence of SEQ ID NO:24, or a functional variant thereof (i.e. wherein the variant TCR Vα domain retains the ability to specifically bind to a Bob1 antigen (e.g. the peptide shown in SEQ ID NO:5) when part of a binding protein described herein). Such functional variants may be naturally occurring, synthetic, or synthetically improved functional variants of SEQ ID NO:24. The term “variant” also encompasses homologues and fragments. Functional variants will typically contain only conservative substitutions of one or more amino acids of SEQ ID NO:24, or substitution, deletion or insertion of non-critical amino acids in non-critical regions of the protein.

Non-functional variants are amino acid sequence variants of SEQ ID NO: 24 that do not specifically bind to a Bob1 antigen (e.g. the peptide shown in SEQ ID NO:5). Non-functional variants will typically contain a non-conservative substitution, a deletion, or insertion or premature truncation of the amino acid sequence of SEQ ID NO:24 or a substitution, insertion or deletion in critical amino acids or critical regions. Methods for identifying functional and non-functional variants are well known to a person of ordinary skill in the art.

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 24, whilst retaining the ability to specifically bind to a Bob1 antigen (e.g. the peptide shown in SEQ ID NO:5). In other words, a functional TCR Vα domain with one or several amino acid substitutions compared to the sequence of SEQ ID NO:24 is also encompassed. As stated previously, the amino acid substitution may be a conservative amino acid substitution. The variability in sequence compared to SEQ ID NO:24 may all be in regions of the TCR Vα domain that do not form CDRs (i.e. the variant may have the CDRs of SEQ ID NO: 12, SEQ ID NO: 6 and/or SEQ ID NO: 9, and still have 25% (or less) sequence variability compared to SEQ ID NO:24). In other words, the sequence of the CDRs of SEQ ID NO: 24 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 24).

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 24, wherein the TCR Vα domain comprises a CDR3 having an amino acid sequence of SEQ ID NO: 12. In this example, the TCR Vα domain CDR1 may have an amino acid sequence of SEQ ID NO: 6 and the TCR Vα domain CDR2 may have an amino acid sequence of SEQ ID NO: 9.

As another example, the encoded TCR Vα domain may comprise an amino acid sequence having at the amino acid sequence of SEQ ID NO: 24, with 0 to 10 (or 0 to 5) amino acid substitutions, insertions or deletions), wherein the TCR Vα domain comprises a CDR3 having an amino acid sequence of SEQ ID NO: 12. In this example, the TCR Vα domain CDR1 may have an amino acid sequence of SEQ ID NO: 6 and the TCR Vα domain CDR2 may have an amino acid sequence of SEQ ID NO: 9.

In examples where the TCR Vα domain has the amino acid sequence of SEQ ID NO:24, the TCR Vα domain may be encoded by the nucleic acid sequence of SEQ ID NO:25 or SEQ ID NO:26, or a genetically degenerate sequence thereof (i.e. other nucleic acid sequences that encode the same protein as a result of the degeneracy of the genetic code). It is noted that SEQ ID NO:26 is the codon optimised version of the nucleic acid sequence for TCR Vα domain of clone 1C5.6 (the non-optimised sequence being SEQ ID NO:25).

For the avoidance of doubt, the nucleic acid sequence encoding the TCR Vα domain may also encode a TCR α chain constant domain. An example of a suitable constant domain is encoded in the MP71-TCR-flex retroviral vector. However, the invention is not limited to this specific constant domain, and encompasses any appropriate TCR α chain constant domain. The constant domain may be murine derived, human derived or humanised. Methods for identifying or generating appropriate constant domains are well known to a person of skill in the art and are well within their routine capabilities.

By way of example only, the constant domain may be encoded by or derived from a vector, such as a lentiviral, retroviral or plasmid vector but also adenovirus, adeno-associated virus, vaccinia virus, canary poxvirus or herpes virus vectors in which murine or human constant domains are pre-cloned. Recently, minicircles have also been described for TCR gene transfer (non-viral Sleeping Beauty transposition from minicircle vectors as published by R Monjezi, et al., 2017). Moreover, naked (synthetic) DNA/RNA can also be used to introduce the TCR. As an example, a pMSGV retroviral vector with pre-cloned TCR-Ca and Cb genes as described in LV Coren et al., BioTechniques 2015 may be used to provide an appropriate constant domain. Alternatively, single stranded or double stranded DNA or RNA can be inserted by homologous directed repair into the TCR locus (see Roth et al 2018 Nature vol 559; page 405). As a further option, non-homologous end joining is possible.

Examples of specific TCR α chain amino acid sequences that include a TCR Vα domain described herein with an appropriate constant domain are shown in SEQ ID NO: 30 and SEQ ID NO: 31. It is noted that the constant domain shown in SEQ ID NO:31 is murine. Appropriate functional variants of SEQ ID NO:30 and SEQ ID NO:31 are also encompassed (e.g. variants having at least 75% (e.g. at least 75%, at least 80%, at least 85%, at least 90%, at least 95% etc) sequence identity to the amino acid sequence of SEQ ID NO: 30 or SEQ ID NO:31, wherein the variant TCR α chain amino acid sequence retains its ability to specifically bind to a Bob1 antigen (e.g. the peptide shown in SEQ ID NO:5) when part of a binding protein described herein). In other words, a functional TCR α chain with one or several amino acid substitutions compared to the sequence of SEQ ID NO:30 or SEQ ID NO:31 is also encompassed. As stated previously, the amino acid substitution may be a conservative amino acid substitution. The variability in sequence compared to SEQ ID NO:30 or SEQ ID NO:31 may all be in regions of the TCR α chain that do not form CDRs (i.e. the variant may have the CDRs of SEQ ID NO: 12, SEQ ID NO: 6 and/or SEQ ID NO: 9, and still have 25% (or less) sequence variability compared to SEQ ID NO:30 or SEQ ID NO:31). In other words, the sequence of the CDRs of SEQ ID NO: 30 or SEQ ID NO:31 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 30 or SEQ ID NO:31 as appropriate).

As an example, the encoded TCR α chain may comprise an amino acid sequence having at least 75% (e.g. at least 75%, at least 80%, at least 85%, at least 90%, at least 95% etc) sequence identity to the amino acid sequence of SEQ ID NO: 30 or SEQ ID NO: 31, wherein the TCR α chain comprises a CDR3 having an amino acid sequence of SEQ ID NO: 12. In this example, the TCR α chain CDR1 may have an amino acid sequence of SEQ ID NO:6 and the TCR α chain CDR2 may have an amino acid sequence of SEQ ID NO: 9.

In examples where the TCR α chain has the amino acid sequence of SEQ ID NO:30, the TCR α chain may be encoded by the nucleic acid sequence of SEQ ID NO:32, or a genetically degenerate sequence thereof (i.e. other nucleic acid sequences that encode the same protein as a result of the degeneracy of the genetic code). It is noted that SEQ ID NO:32 is the nucleic acid sequence for TCR Vα domain of clone 1C5.6.

In examples where the TCR α chain has the amino acid sequence of SEQ ID NO:31, the TCR α chain may be encoded by the nucleic acid sequence of SEQ ID NO:33, or a genetically degenerate sequence thereof (i.e. other nucleic acid sequences that encode the same protein as a result of the degeneracy of the genetic code).

### Components of the TCR β Chain Variable (Vβ) Domain

The isolated nucleic acid composition described herein encodes a Bob1 antigen-specific binding protein. The encoded Bob1 antigen-specific binding protein comprises a TCR Vα domain comprising a CDR3 amino acid sequence having at least 80% sequence identity to SEQ ID NO: 12 as described above. The encoded Bob1 antigen-specific binding protein also comprises a TCR Vβ domain comprising a CDR3 amino acid sequence having at least 80% sequence identity to SEQ ID NO: 21.

An example of an appropriate TCR Vβ domain CDR3 amino acid sequence that confers specific binding to a Bob1 antigen is shown in SEQ ID NO:21. As would be clear to a person of skill in the art, variants of the amino acid sequence shown in SEQ ID NO:21 may also be functional (i.e. retain their ability to confer specific binding to a Bob1 antigen (e.g. the peptide shown in SEQ ID NO:5) when the CDR3 is part of TCR Vβ domain). Such functional variants are therefore encompassed herein.

For example, appropriate (functional) Vβ domain CDR3 amino acid sequences may have at least 80% sequence identity to SEQ ID NO: 21, i.e. they may have at least 80%, at least 84%, at least 90%, at least 91%, at least 92%, at least 93%, at least 94%, at least 95%, at least 96%, at least 97%, at least 98%, at least 99%, or 100% sequence identity to SEQ ID NO: 21. Suitably, percent identity is calculated as the percentage of identity to the entire length of the reference sequence (e.g. SEQ ID NO:21). In other words, appropriate (functional) Vβ domain CDR3 amino acid sequences may vary from the sequence shown in SEQ ID NO:21 by one or several (e.g. two) amino acids. As stated above, functional variants of SEQ ID NO:21 retain their ability to confer specific binding to a Bob1 antigen (e.g. the peptide shown in SEQ ID NO:5) when the CDR3 is part of TCR Vβ domain.

Functional variants may be naturally occurring, synthetic, or synthetically improved functional variants of SEQ ID NO:21. The term “variant” also encompasses homologues and fragments. Functional variants will typically contain only conservative substitutions of one or more amino acids of SEQ ID NO:21, or substitution, deletion or insertion of non-critical amino acids in non-critical regions of the CDR3.

Non-functional variants are amino acid sequence variants of SEQ ID NO:21 that do not specifically bind to a Bob1 antigen (e.g. the peptide shown in SEQ ID NO:5). Non-functional variants will typically contain a non-conservative substitution, a deletion, or insertion or premature truncation of the amino acid sequence of SEQ ID NO:21 or a substitution, insertion or deletion in critical amino acids or critical regions. Methods for identifying functional and non-functional variants are well known to a person of ordinary skill in the art.

In one example, the CDR3 of the Vβ domain comprises or consists of the amino acid sequence of SEQ ID NO: 21. In examples where the TCR Vβ domain CDR3 has the amino acid sequence of SEQ ID NO:21, the CDR3 may be encoded by the nucleic acid sequence of SEQ ID NO:22 or SEQ ID NO:23, or a genetically degenerate sequence thereof (i.e. other nucleic acid sequences that encode the same protein as a result of the degeneracy of the genetic code). It is noted that SEQ ID NO:23 is the codon optimised version of the nucleic acid sequence for CDR3 of clone 1C5.6 (the non-optimised sequence being SEQ ID NO:22).

The encoded TCR Vβ domain may comprise, in addition to the specified CDR3, a CDR1 comprising an amino acid sequence of SEQ ID NO: 15, or a functional variant thereof (i.e. wherein the variant retains the ability to specifically bind to the Bob1 antigen (e.g. the peptide shown in SEQ ID NO:5)). Such functional variants may be naturally occurring, synthetic, or synthetically improved functional variants of SEQ ID NO: 15. The term “variant” also encompasses homologues and fragments. Functional variants will typically contain only conservative substitutions of one or more amino acids of SEQ ID NO: 15, or substitution, deletion or insertion of non-critical amino acids in non-critical regions of the protein.

Non-functional variants are amino acid sequence variants of SEQ ID NO: 15 that do not specifically bind to the Bob1 antigen (e.g. the peptide shown in SEQ ID NO:5). Non-functional variants will typically contain a non-conservative substitution, a deletion, or insertion or premature truncation of the amino acid sequence of SEQ ID NO: 15 or a substitution, insertion or deletion in critical amino acids or critical regions. Methods for identifying functional and non-functional variants are well known to a person of ordinary skill in the art.

For example, appropriate functional Vβ domain CDR1 amino acid sequences may have at least 80% sequence identity to SEQ ID NO: 15, i.e. it may have at least 80%, at least 83%, at least 85%, at least 90%, at least 91%, at least 92%, at least 93%, at least 94%, at least 95%, at least 96%, at least 97%, at least 98%, at least 99%, or 100% sequence identity to SEQ ID NO: 15. Suitably, percent identity is calculated as the percentage of identity to the entire length of the reference sequence (e.g. SEQ ID NO:15). In other words, appropriate (functional) Vβ domain CDR1 amino acid sequences may vary from the sequence shown in SEQ ID NO:15 by one or several amino acids. As stated previously, the variant may comprise an amino acid substitution such as a conservative amino acid substitution compared to the sequence shown in SEQ ID NO:15). As stated above, functional variants of SEQ ID NO: 15 retain the ability to specifically bind to the Bob1 antigen (e.g. the peptide shown in SEQ ID NO:5) when the CDR1 is part of TCR Vβ domain).

In one example, the CDR1 of the Vβ domain comprises or consists of the amino acid sequence of SEQ ID NO: 15. In examples where the TCR Vα domain CDR1 has the amino acid sequence of SEQ ID NO:15, the CDR1 may be encoded by the nucleic acid sequence of SEQ ID NO:16 or SEQ ID NO:17, or a genetically degenerate sequence thereof (i.e. other nucleic acid sequences that encode the same protein as a result of the degeneracy of the genetic code). It is noted that SEQ ID NO:17 is the codon optimised version of the nucleic acid sequence for CDR1 of clone 1C5.6 (the non-optimised sequence being SEQ ID NO:16).

The encoded TCR Vβ domain may also comprise, in addition to the specified CDR3 (and optionally the specified CDR1 above), a CDR2 having an amino acid sequence of SEQ ID NO: 18, or a functional variant thereof (i.e. wherein the variant retains the ability to specifically bind to HLA-B*35:01). Such functional variants may be naturally occurring, synthetic, or synthetically improved functional variants of SEQ ID NO:18. The term “variant” also encompasses homologues and fragments. Functional variants will typically contain only conservative substitutions of one or more amino acids of SEQ ID NO:18, or substitution, deletion or insertion of non-critical amino acids in non-critical regions of the protein.

Non-functional variants are amino acid sequence variants of SEQ ID NO:18 that do not specifically bind to HLA-B*35:01. Non-functional variants will typically contain a non-conservative substitution, a deletion, or insertion or premature truncation of the amino acid sequence of SEQ ID NO:18 or a substitution, insertion or deletion in critical amino acids or critical regions. Methods for identifying functional and non-functional variants are well known to a person of ordinary skill in the art.

For example, appropriate functional Vβ domain CDR2 amino acid sequences may have at least 80% sequence identity to SEQ ID NO: 18, i.e. it may have at least 80%, at least 83%, at least 85%, at least 90%, at least 91%, at least 92%, at least 93%, at least 94%, at least 95%, at least 96%, at least 97%, at least 98%, at least 99%, or 100% sequence identity to SEQ ID NO: 18. Suitably, percent identity is calculated as the percentage of identity to the entire length of the reference sequence (e.g. SEQ ID NO:18). In other words, appropriate (functional) Vβ domain CDR2 amino acid sequences may vary from the sequence shown in SEQ ID NO:18 by one or several amino acids. As stated previously, the variant may comprise an amino acid substitution such as a conservative amino acid substitution compared to the sequence shown in SEQ ID NO:18). As stated above, a functional variant of SEQ ID NO: 18 retains the ability to specifically bind to HLA-B*35:01.

In one example, the CDR2 of the Vβ domain comprises or consists of the amino acid sequence of SEQ ID NO: 18. In examples where the TCR Vβ domain CDR2 has the amino acid sequence of SEQ ID NO:18, the CDR2 may be encoded by the nucleic acid sequence of SEQ ID NO:19 or SEQ ID NO:20, or a genetically degenerate sequence thereof (i.e. other nucleic acid sequences that encode the same protein as a result of the degeneracy of the genetic code). It is noted that SEQ ID NO:20 is the codon optimised version of the nucleic acid sequence for CDR2 of clone 1C5.6 (the non-optimised sequence being SEQ ID NO:19).

The encoded TCR Vβ domain may therefore comprise the CDRs mentioned in detail above (by SEQ ID specifically i.e. SEQ ID NO:21, SEQ ID NO: 15 and SEQ ID NO: 18, or functional variants thereof), with appropriate intervening sequences between the CDRs.

The encoded TCR Vβ domain may have an amino acid sequence of SEQ ID NO:27, or a functional variant thereof (i.e. wherein the variant TCR Vβ domain retains the ability to specifically bind to a Bob1 antigen (e.g. the peptide shown in SEQ ID NO:5) when part of a binding protein described herein). Such functional variants may be naturally occurring, synthetic, or synthetically improved functional variants of SEQ ID NO:27. The term “variant” also encompasses homologues and fragments. Functional variants will typically contain only conservative substitutions of one or more amino acids of SEQ ID NO:27, or substitution, deletion or insertion of non-critical amino acids in non-critical regions of the protein.

Non-functional variants are amino acid sequence variants of SEQ ID NO: 27 that do not specifically bind to a Bob1 antigen (e.g. the peptide shown in SEQ ID NO:5). Non-functional variants will typically contain a non-conservative substitution, a deletion, or insertion or premature truncation of the amino acid sequence of SEQ ID NO:27 or a substitution, insertion or deletion in critical amino acids or critical regions. Methods for identifying functional and non-functional variants are well known to a person of ordinary skill in the art.

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 27, whilst retaining the ability to specifically bind to a Bob1 antigen (e.g. the peptide shown in SEQ ID NO:5). In other words, a functional TCR Vβ domain with one or several amino acid substitutions compared to the sequence of SEQ ID NO:27 is also encompassed. As stated previously, the amino acid substitution may be a conservative amino acid substitution. The variability in sequence compared to SEQ ID NO:27 may all be in regions of the TCR Vβ domain that do not form CDRs (i.e. the variant may have the CDRs of SEQ ID NO: 21, SEQ ID NO: 15 and/or SEQ ID NO: 18, and still have 25% (or less) sequence variability compared to SEQ ID NO:27). In other words, the sequence of the CDRs of SEQ ID NO: 27 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 27).

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 27, wherein the TCR Vβ domain comprises a CDR3 having an amino acid sequence of SEQ ID NO: 21. In this example, the TCR Vβ domain CDR1 may have an amino acid sequence of SEQ ID NO:15 and the TCR Vβ domain CDR2 may have an amino acid sequence of SEQ ID NO: 18.

In examples where the TCR Vβ domain has the amino acid sequence of SEQ ID NO:27, the TCR Vβ domain may be encoded by the nucleic acid sequence of SEQ ID NO:28 or SEQ ID NO:29, or a genetically degenerate sequence thereof (i.e. other nucleic acid sequences that encode the same protein as a result of the degeneracy of the genetic code). It is noted that SEQ ID NO:29 is the codon optimised version of the nucleic acid sequence for TCR Vβ domain of clone 1C5.6 (the non-optimised sequence being SEQ ID NO:28).

For the avoidance of doubt, the nucleic acid sequence encoding the TCR Vβ domain may also encode a TCR β chain constant domain. An example of a suitable constant domain is encoded in the MP71-TCR-flex retroviral vector. However, the invention is not limited to this specific constant domain and encompasses any appropriate TCR β chain constant domain. The constant domain may be murine derived, human derived or humanised. Methods for identifying or generating appropriate constant domains are well known to a person of skill in the art and are well within their routine capabilities.

By way of example only, the constant domain may be encoded by or derived from a vector, such as a lentiviral, retroviral or plasmid vector but also adenovirus, adeno-associated virus, vaccinia virus, canary poxvirus or herpes virus vectors in which murine or human constant domains are pre-cloned. Recently, minicircles have also been described for TCR gene transfer (non-viral Sleeping Beauty transposition from minicircle vectors as published by R Monjezi et al., Leukemia 2017). Moreover, naked (synthetic) DNA/RNA can also be used to introduce the TCR. As an example, a pMSGV retroviral vector with pre-cloned TCR-Ca and Cb genes as described in L V Coren et al., BioTechniques 2015 may be used to provide an appropriate constant domain.

Alternatively, single stranded or double stranded DNA or RNA can be inserted by homologous directed repair into the TCR locus (see Roth et al 2018 Nature vol 559; page 405). As a further option, non-homologous end joining is possible.

Examples of specific TCR β chain amino acid sequences that include a TCR Vβ domain described herein and an appropriate constant domain are shown in SEQ ID NO: 34 and SEQ ID NO: 35. It is noted that the constant domain shown in SEQ ID NO:35 is murine. Appropriate functional variants of SEQ ID NO:34 and SEQ ID NO:35 are also encompassed (e.g. variants having at least 75% (e.g. at least 75%, at least 80%, at least 85%, at least 90%, at least 95% etc) sequence identity to the amino acid sequence of SEQ ID NO: 34 or SEQ ID NO:35, wherein the variant TCR β chain amino acid sequence retains its ability to specifically bind to a Bob1 antigen (e.g. the peptide shown in SEQ ID NO:5) when part of a binding protein described herein). In other words, a functional TCR β chain with one or several amino acid substitutions compared to the sequence of SEQ ID NO: 34 or SEQ ID NO:35 is also encompassed. As stated previously, the amino acid substitution may be a conservative amino acid substitution. The variability in sequence compared to SEQ ID NO:34 or SEQ ID NO:35 may all be in regions of the TCR β chain that do not form CDRs (i.e. the variant may have the CDRs of SEQ ID NO: 21, SEQ ID NO: 15 and/or SEQ ID NO: 18, and still have 25% (or less) sequence variability compared to SEQ ID NO:34 or SEQ ID NO:35. In other words, the sequence of the CDRs of SEQ ID NO: 34 or SEQ ID NO:35 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 34 or SEQ ID NO:35 as appropriate).

As an example, the encoded TCR β chain may comprise an amino acid sequence having at least 75% (e.g. at least 75%, at least 80%, at least 85%, at least 90%, at least 95% etc) sequence identity to the amino acid sequence of SEQ ID NO: 34 or SEQ ID NO: 35, wherein the TCR β chain comprises a CDR3 having an amino acid sequence of SEQ ID NO: 21. In this example, the TCR β chain CDR1 may have an amino acid sequence of SEQ ID NO: 15 and the TCR β chain CDR2 may have an amino acid sequence of SEQ ID NO: 18.

In examples where the TCR β chain has the amino acid sequence of SEQ ID NO:34, the TCR β chain may be encoded by the nucleic acid sequence of SEQ ID NO:36, or a genetically degenerate sequence thereof (i.e. other nucleic acid sequences that encode the same protein as a result of the degeneracy of the genetic code). It is noted that SEQ ID NO:36 is the nucleic acid sequence for TCR Vβ domain of clone 1C5.6.

In examples where the TCR β chain has the amino acid sequence of SEQ ID NO:35, the TCR β chain may be encoded by the nucleic acid sequence of SEQ ID NO:37, or a genetically degenerate sequence thereof (i.e. other nucleic acid sequences that encode the same protein as a result of the degeneracy of the genetic code).

In a particular example, a nucleic acid composition described herein encodes a Bob1 antigen-specific binding protein having a TCR Vα domain with a CDR3 amino acid sequence comprising or consisting of the amino acid sequence of SEQ ID NO: 12; and a TCR Vβ domain with a CDR3 comprising or consisting of the amino acid sequence of SEQ ID NO:21. In addition, the Bob1 antigen may comprise or consist of the sequence shown in SEQ ID NO:5. Furthermore, the TCR Vα domain may be part of a TCR α chain having a constant domain and the TCR Vβ domain may be part of a TCR β chain having a constant domain.

In this particular example, the CDR3 of the Vα domain may be encoded by a nucleic acid sequence comprising the sequence of SEQ ID NO: 13 or SEQ ID NO:14; and the CDR3 of the Vβ domain may be encoded by a nucleic acid sequence comprising the sequence of SEQ ID NO: 22 or SEQ ID NO:23.

In this particular example, the Vα domain may comprise an amino acid sequence having at least 80% sequence identity to, comprising, or consisting of, SEQ ID NO: 24; and the Vβ domain may comprise an amino acid sequence having at least 80% sequence identity to, comprising, or consisting of, SEQ ID NO: 27. In one example, the Vα domain comprises the amino acid sequence of SEQ ID NO: 24 and the Vβ domain comprises the amino acid sequence of SEQ ID NO: 27. In such cases, the Vα domain may be encoded by a nucleic acid sequence comprising the sequence of SEQ ID NO: 25 or SEQ ID NO: 26; and the Vβ domain may be encoded by a nucleic acid sequence comprising the sequence of SEQ ID NO: 28 or SEQ ID NO:29.

In this particular example, the TCR Vα domain may include a CDR1 amino acid sequence comprising or consisting of the amino acid sequence of SEQ ID NO:6 and a CDR2 amino acid sequence comprising or consisting of the amino acid sequence of SEQ ID NO:9. Furthermore, the TCR Vβ domain may include a CDR1 amino acid sequence comprising or consisting of the amino acid sequence of SEQ ID NO:15 and a CDR2 amino acid sequence comprising or consisting of the amino acid sequence of SEQ ID NO: 18.

For the avoidance of doubt, this particular example encompasses components of TCR clone 1C5.6 exemplified herein. The different components of TCR clone 1C5.6 and their respective SEQ ID Nos are summarised in Table 1 below.

TABLE 1
component parts of clone 1C5.6 with their respective SEQ ID Nos.

| SEQ ID NO | TCR COMPONENT | AA or NT |
|---|---|---|
| 6 | α CDR1 | AA |
| 7 | α CDR1 | NT |
| 8 | α CDR1 | NT co |
| 9 | α CDR2 | AA |
| 10 | α CDR2 | NT |
| 11 | α CDR2 | NT co |
| 12 | α CDR3 | AA |
| 13 | α CDR3 | NT |
| 14 | α CDR3 | NT co |
| 15 | β CDR1 | AA |
| 16 | β CDR1 | NT |
| 17 | β CDR1 | NT co |
| 18 | β CDR2 | AA |
| 19 | β CDR2 | NT |
| 20 | β CDR2 | NT co |
| 21 | β CDR3 | AA |
| 22 | β CDR3 | NT |
| 23 | β CDR3 | NT co |
| 24 | α VJ | AA |
| 25 | α VJ | NT |
| 26 | α VJ | NT co |
| 27 | β VDJ | AA |
| 28 | β VDJ | NT |
| 29 | β VDJ | NT co |
| 30 | α VJ and constant | AA |
| 31 | α VJ and constant (murine) | AA |
| 32 | α VJ and constant | NT |
| 33 | α VJ and constant (murine) | NT co |
| 34 | β VDJ and constant | AA |
| 35 | β VDJ and constant (murine) | AA |
| 36 | β VDJ and constant | NT |
| 37 | β VDJ and constant (murine) | NT co |

As stated in more detail elsewhere herein, the nucleic acid composition described herein encodes both a TCR Vα domain and a TCR Vβ domain, which form the binding protein that is capable of specifically binding to the Bob1 antigen. In examples where the TCR Vα domain and the TCR Vβ domain are encoded by the same nucleic acid sequence, the TCR Vα domain and TCR Vβ domain may be joined together via a linker, e.g. a linker that enables expression of two proteins or polypeptides from the same vector. By way of example, a linker comprising a porcine teschovirus-1 2A (P2A) sequence may be used, such as 2A sequences from foot-and-mouth disease virus (F2A), equine rhinitis A virus (E2A) or Thosea asigna virus (T2A) as published by A. L. Szymczak et al., Nature Biotechnology 22, 589-594 (2004) or 2A-like sequences. 2A and 2A-like sequences are linkers that are cleavable once the nucleic acid molecule has been transcribed and translated. Another example of a linker is an internal ribosomal entry sites (IRES) which enables translation of two proteins or polypeptides from the same transcript. Any other appropriate linker may also be used. As a further example, the nucleic acid sequence encoding the TCR Vα domain and nucleic acid sequence encoding the TCR Vβ domain may be cloned into a vector with dual internal promoters (see e.g. S Jones et al., Human Gene Ther 2009). The identification of appropriate linkers and vectors that enable expression of both the TCR Vα domain and the TCR Vβ domain is well within the routine capabilities of a person of skill in the art.

Additional appropriate polypeptide domains may also be encoded by the nucleic acid sequences that encode the TCR Vα domain and/or the TCR Vβ domain. By way of example only, the nucleic acid sequence may comprise a membrane targeting sequence that provides for transport of the encoded polypeptide to the cell surface membrane of the modified cell. Other appropriate additional domains are well known and are described, for example, in WO2016/071758.

In one example, the nucleic acid composition described herein may encode a soluble TCR. For example, the nucleic acid composition may encode the variable domain of the TCR alpha and beta chains respectively together with an immune-modulator molecule such as a CD3 agonist (e.g. an anti-CD3 scFv). The CD3 antigen is present on mature human T cells, thymocytes and a subset of natural killer cells. It is associated with the TCR and is involved in signal transduction of the TCR. Antibodies specific for the human CD3 antigen are well known. One such antibody is the murine monoclonal antibody OKT3, which is the first monoclonal antibody approved by the FDA. Other antibodies specific for CD3 have also been reported (see e.g. WO2004/106380; U.S. Patent Application Publication No. 2004/0202657; U.S. Pat. No. 6,750,325). Immune mobilising mTCR Against Cancer (ImmTAC; Immunocore Limited, Milton Partk, Abington, Oxon, United Kingdom) are bifunctional proteins that combine affinity monoclonal T cell receptor (mTCR) targeting with a therapeutic mechanism of action (i.e., an anti-CD3 scFv). In another example, a soluble TCR of the invention may be combined with a radioisotope or a toxic drug. Appropriate radioisotopes and/or toxic drugs are well known in the art and are readily identifiable by a person of ordinary skill in the art.

In one example, the nucleic acid composition may encode a chimeric single chain TCR wherein the TCR alpha chain variable domain is linked to the TCR beta chain variable domain and a constant domain which is e.g. fused to the CD3 zeta signalling domain. In this example, the linker is non-cleavable. In an alternative embodiment, the nucleic acid composition may encode a chimeric two chain TCR in which the TCR alpha chain variable domain and the TCR beta chain variable domain are each linked to a CD3 zeta signalling domain or other transmembrane and intracellular domains. Methods for preparing such single chain TCRs and two chain TCRs are well known in the art; see for example R A Willemsen et al, Gene Therapy 2000.

### Vector Systems

A vector system is also provided which includes a nucleic acid composition described herein. The vector system may have one or more vectors. As discussed previously, the binding protein components that are encoded by the nucleic acid composition may be encoded by one or more nucleic acid sequences in the nucleic acid composition. In examples where all of the binding protein components are encoded by a single nucleic acid sequence, the nucleic acid sequence may be present within a single vector (and thus the vector system described herein may comprise of one vector only). In examples where the binding protein components are encoded by two or more nucleic acid sequences (wherein the plurality of nucleic acid sequences, together, encode all of the components of the binding protein) these two or more nucleic acid sequences may be present within one vector (e.g. in different open reading frames of the vector), or may be distributed over two or more vectors. In this example, the vector system will comprise a plurality of distinct vectors (i.e. vectors with different nucleotide sequences).

Any appropriate vector can be used. By way of example only, the vector may be a plasmid, a cosmid, or a viral vector, such as a retroviral vector or a lentiviral vector. Adenovirus, adeno-associated virus, vaccinia virus, canary poxvirus, herpes virus, minicircle vectors and naked (synthetic) DNA/RNA may also be used (for details on minicircle vectors, see for example non-viral Sleeping Beauty transposition from minicircle vectors as published by R Monjezi et al., Leukemia 2017). Alternatively, single stranded or double stranded DNA or RNA can be used to transfect lymphocytes with a TCR of interest (see Roth et al 2018 Nature vol 559; page 405).

As used herein, the term “vector” refers to a nucleic acid sequence capable of transporting another nucleic acid sequence to which it has been operably linked. The vector can be capable of autonomous replication or it can integrate into a host DNA. The vector may include restriction enzyme sites for insertion of recombinant DNA and may include one or more selectable markers or suicide genes. The vector can be a nucleic acid sequence in the form of a plasmid, a bacteriophage or a cosmid. Preferably the vector is suitable for expression in a cell (i.e. the vector is an “expression vector”). Preferably, the vector is suitable for expression in a human T cell such as a CD8⁺ T cell or CD4⁺ T cell, or stem cell, iPS cell, or NK cell. In certain aspects, the vector is a viral vector, such as a retroviral vector, a lentiviral vector or an adeno-associated vector. Optionally, the vector is selected from the group consisting of an adenovirus, vaccinia virus, canary poxvirus, herpes virus, minicircle vector and synthetic DNA or synthetic RNA.

Preferably the (expression) vector is capable of propagation in a host cell and is stably transmitted to future generations.

The vector may comprise regulatory sequences. “Regulatory sequences” as used herein, refers to, DNA or RNA elements that are capable of controlling gene expression. Examples of expression control sequences include promoters, enhancers, silencers, TATA-boxes, internal ribosomal entry sites (IRES), attachment sites for transcription factors, transcriptional terminators, polyadenylation sites etc. Optionally, the vector includes one or more regulatory sequences operatively linked to the nucleic acid sequence to be expressed. Regulatory sequences include those which direct constitutive expression, as well as tissue-specific regulatory and/or inducible sequences.

Optionally, the vector comprises the nucleic acid sequence of interest operably linked to a promoter. “Promoter”, as used herein, refers to the nucleotide sequences in DNA to which RNA polymerase binds to start transcription. The promoter may be inducible or constitutively expressed. Alternatively, the promoter is under the control of a repressor or stimulatory protein. The promoter may be one that is not naturally found in the host cell (e.g. it may be an exogenous promoter). The skilled person in the art is well aware of appropriate promoters for use in the expression of target proteins, wherein the selected promoter will depend on the host cell.

“Operably linked” refers to a single or a combination of the below-described control elements together with a coding sequence in a functional relationship with one another, for example, in a linked relationship so as to direct expression of the coding sequence.

The vector may comprise a transcriptional terminator. “Transcriptional terminator” as used herein, refers to a DNA element, which terminates the function of RNA polymerases responsible for transcribing DNA into RNA. Preferred transcriptional terminators are characterized by a run of T residues preceded by a GC rich dyad symmetrical region.

The vector may comprise a translational control element. “Translational control element”, as used herein, refers to DNA or RNA elements that control the translation of mRNA. Preferred translational control elements are ribosome binding sites. Preferably, the translational control element is from a homologous system as the promoter, for example a promoter and its associated ribozyme binding site. Preferred ribosome binding sites are known, and will depend on the chosen host cell.

The vector may comprise restriction enzyme recognition sites. “Restriction enzyme recognition site” as used herein, refers to a motif on the DNA recognized by a restriction enzyme.

The vector may comprise a selectable marker. “Selectable marker” as used herein, refers to proteins that, when expressed in a host cell, confer a phenotype onto the cell which allows a selection of the cell expressing said selectable marker gene. Generally this may be a protein that confers a new beneficial property onto the host cell (e.g. antibiotic resistance) or a protein that is expressed on the cell surface and thus accessible for antibody binding. Appropriate selectable markers are well known in the art.

Optionally, the vector may also comprise a suicide gene. “Suicide gene” as used herein, refers to proteins that induce death of the modified cell upon treatment with specific drugs. By way of example, suicide can be induced of cells modified by the herpes simplex virus thymidine kinase gene upon treatment with specific nucleoside analogs including ganciclovir, cells modified by human CD20 upon treatment with anti-CD20 monoclonal antibody and cells modified with inducible Caspase9 (iCasp9) upon treatment with AP1903 (reviewed by B S Jones, L S Lamb, F Goldman, A Di Stasi; Improving the safety of cell therapy products by suicide gene transfer. Front Pharmacol. (2014) 5:254). Appropriate suicide genes are well known in the art.

Preferably the vector comprises those genetic elements which are necessary for expression of the binding proteins described herein by a host cell. The elements required for transcription and translation in the host cell include a promoter, a coding region for the protein(s) of interest, and a transcriptional terminator.

A person of skill in the art will be well aware of the molecular techniques available for the preparation of (expression) vectors and how the (expression) vectors may be transduced or transfected into an appropriate host cell (thereby generating a modified cell described further below). The (expression) vector system described herein can be introduced into cells by conventional techniques such as transformation, transfection or transduction. “Transformation”, “transfection” and “transduction” refer generally to techniques for introducing foreign (exogenous) nucleic acid sequences into a host cell, and therefore encompass methods such as electroporation, microinjection, gene gun delivery, transduction with retroviral, lentiviral or adeno-associated vectors, lipofection, superfection etc. The specific method used typically depends on both the type of vector and the cell. Appropriate methods for introducing nucleic acid sequences and vectors into host cells such as human cells are well known in the art; see for example Sambrook et al (1989) Molecular Cloning, A Laboratory Manual, Cold Spring Harbor Laboratory, Cold Spring Harbor, N.Y; Ausubel et al (1987) Current Protocols in Molecular Biology, John Wiley and Sons, Inc., NY; Cohen et al (1972) Proc. Natl. Acad. Sci. USA 69, 2110; Luchansky et al (1988) Mol. Microbiol. 2, 637-646. Further conventional methods that are suitable for preparing expression vectors and introducing them into appropriate host cells are described in detail in WO2016/071758 for example.

It is understood that it some examples, the host cell is contacted with the vector system (e.g. viral vector) in vitro, ex vivo, and in some examples, the host cell is contacted with the vector system (e.g. viral vector) in vivo.

The term “host cell” includes any cell into which the nucleic acid composition or vector system described herein may be introduced. Once a nucleic acid molecule or vector system has been introduced into the cell, it may be referred to as a “modified cell” herein. Once the nucleic acid molecule or vector is introduced into the host cell, the resultant modified cell should be capable of expressing the encoded binding protein (and e.g. correctly localising the encoded binding protein for its intended function e.g. transporting the encoded binding protein to the cell surface).

The nucleic acid composition or vector system may be introduced into the cell using any conventional method known in the art. For example, the nucleic acid composition or vector system may be introduced using CRISPR technology. Insertion of the nucleic acid sequences at the endogenous TCR locus by engineering with CRISPR/Cas9 and homologous directed repair (HDR) or non-homologous end joining (NHEJ) is therefore encompassed. Other conventional methods such as transfection, transduction or transformation of the cell may also be used.

The term “modified cell” refers to a genetically altered (e.g. recombinant) cell. The modified cell includes at least one exogenous nucleic acid sequence (i.e. a nucleic acid sequence that is not naturally found in the host cell). In the context of the invention, the exogenous sequence comprises at least one of the T cell receptor component parts described herein for clone 1C5.6 (e.g. the sequences etc that encode the CDR3 sequences that are specific for the Bob1 antigen (e.g. the peptide of SEQ ID NO:5)).

The term “modified cell” refers to the particular subject cell and also to the progeny or potential progeny of such a cell. Because certain modifications may occur in succeeding generations due to either mutation or environmental influences, such progeny may not, in fact, be identical to the parent cell, but are still included within the scope of the term as used herein.

The host cell (and thus the modified cell) is typically a eukaryotic cell, and particularly a human cell (e.g. a T cell such as a CD8⁺ T cell or a CD4⁺ T cell, or a mixture thereof, or a hematopoietic stem cell, an iPSC, or gamma-delta T cell, or NK cell). The host cell (and thus the modified cell) may be an autologous or allogeneic cell (e.g. such as a CD8⁺ T cell or a CD4⁺ T cell, or a mixture thereof, or a hematopoietic stem cell, an iPSC, or gamma-delta T cell, or NK cell). “Allogeneic cell” refers to a cell derived from the different individual to the individual to which it is later administered. In other words, the host cell (and thus the modified cell) may be an isolated cell from a distinct individual compared to the subject to be treated. “Autologous cell” refers to a cell derived from the individual to which it is also later administered. In other words, the host cell (and thus the modified cell) may be an isolated cell from the subject that is to be treated.

The host cell (and thus the modified cell) may be any cell that is able to confer anti-tumour immunity after TCR gene transfer. Non limiting examples of appropriate cells include autologous or allogeneic a CD8 T cell, a CD4 T cell, Natural Killer (NK) cells, NKT cells, gamma-delta T cells, inducible pluripotent stem cells (iPSCs), hematopoietic stem cells or other progenitor cells and any other autologous or allogeneic cell or cell line (NK-92 for example or T cell lines) that is able to confer anti-tumor immunity after TCR gene transfer.

In the context of the methods of treatment described herein, the host cell (and thus the modified cell) is typically for administration to an HLA-B*35:01 positive human subject. In view of this, the host cell (and thus the modified cell) is typically HLA-B*35:01 positive but needs to be Bob1 negative (i.e. modified cells can either beHLA-B*35:01 positive or negative).

In the context of the methods of treatment described herein, the host cell (and thus the modified cell) that is to be administered to the subject can either be autologous or allogeneic.

Advantageously, the modified cell is capable of expressing the binding protein encoded by the nucleic acid composition or vector system described herein (i.e. the TCR component parts) such that the modified cell provides an immunotherapy that specifically targets cells that express Bob1, and thus can be used to treat or prevent hyperproliferative diseases or conditions in a HLA-B*35:01 positive human subject, for example, Bob1 expressing B cell malignancies or multiple myeloma. More details on this use are given below.

### Pharmaceutical Compositions

A nucleic acid composition, vector system or modified cell described herein may be provided as part of a pharmaceutical composition. Advantageously, such compositions may be administered to a human subject in need thereof (as described elsewhere herein).

A pharmaceutical composition may comprise a nucleic acid composition, vector system or modified cell described herein along with a pharmaceutically acceptable excipient, adjuvant, diluent and/or carrier.

Compositions may routinely contain pharmaceutically acceptable concentrations of salt, buffering agents, preservatives, compatible carriers, supplementary immune potentiating agents such as adjuvants and cytokines and optionally other therapeutic agents or compounds.

As used herein, “pharmaceutically acceptable” refers to a material that is not biologically or otherwise undesirable, i.e., the material may be administered to an individual along with the selected nucleic acid composition, vector system or modified cell without causing any undesirable biological effects or interacting in a deleterious manner with any of the other components of the pharmaceutical composition in which it is contained.

Excipients are natural or synthetic substances formulated alongside an active ingredient (e.g. a nucleic acid sequence, vector, modified cell or isolated peptide as provided herein), included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, glycerol, ethanol, and the like.

Adjuvants are pharmacological and/or immunological agents that modify the effect of other agents in a formulation. Pharmaceutically acceptable adjuvants are well known in the art. A suitable adjuvant is therefore easily identifiable by one of ordinary skill in the art.

Diluents are diluting agents. Pharmaceutically acceptable diluents are well known in the art. A suitable diluent is therefore easily identifiable by one of ordinary skill in the art.

Carriers are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art. A suitable carrier is therefore easily identifiable by one of ordinary skill in the art.

### Treatment of a Subject

Pharmaceutical compositions described herein may advantageously be administered to a HLA-B*35:01 positive human subject in need thereof.

Typically, the subject in need of treatment has a disease or condition that is associated with an elevated level of Bob1. The disease or condition may be a hyperproliferative disease or condition. For example, the disease or condition may be a Bob1 expressing tumor or cancer.

In one example, the pharmaceutical composition may be for use in inducing or enhancing an immune response (e.g. a cell mediated response) in an HLA-B*35:01 positive human subject diagnosed with a hyperproliferative disease or condition (e.g. a targeted immune response to malignant cells that present the Bob1-HLA-B*35:01 restricted peptide).

The phrase “induced or enhanced immune response” refers to an increase in the immune response (e.g. a cell mediated immune response such as a T cell mediated immune response) of the subject during or after treatment compared to their immune response prior to treatment. An “induced or enhanced” immune response therefore encompasses any measurable increase in the immune response that is directly or indirectly targeted to the hyperproliferative disease or condition being treated (or prevented).

In another example, the pharmaceutical composition may be for use in stimulating a cell mediated immune response to a target cell population or tissue in an HLA-B*35:01 positive human subject. In such an example, the target cell population or tissue may be a Bob1 expressing target cell population or tissue. Typically, it is a Bob1 expressing malignant target cell population or tissue. For example, it may be a target cell population or tissue comprising a Bob1 expressing tumor or cancer.

The pharmaceutical composition may also be for use in providing anti-tumor immunity to an HLA-B*35:01 positive human subject.

In another example, the pharmaceutical composition may be for use in treating an HLA-B*35:01 positive human subject having a disease or condition associated with an elevated level of Bob1. Typically, the disease or condition associated with an elevated level of Bob1 may be a hyperproliferative disease or condition.

A person of skill in the art will be fully aware of hyperproliferative diseases or conditions that may be treated in accordance with the invention. By way of example, appropriate hyperproliferative diseases or conditions include a B cell malignancy or multiple myeloma (particularly, Bob1 expressing B cell malignancy or multiple myeloma). In one example, the B cell malignancy may be a B cell lymphoma or a B cell leukemia. For example, the B cell malignancy may be selected from the group consisting of mantle cell lymphoma, acute lymphoblastic leukemia, chronic lymphocytic leukemia, follicular lymphoma and large B cell lymphoma.

As would be clear to a person skilled in the art, the hyperproliferative diseases or conditions may comprise at least one tumor (particularly, at least one Bob1 expressing tumor).

As used herein, the terms “treat”, “treating” and “treatment” are taken to include an intervention performed with the intention of preventing the development or altering the pathology of a condition, disorder or symptom (e.g. a hyperproliferative disease or condition). Accordingly, “treatment” refers to both therapeutic treatment and prophylactic or preventative measures, wherein the object is to prevent or slow down (lessen) the targeted condition, disorder or symptom. “Treatment” therefore encompasses a reduction, slowing or inhibition of the amount or concentration of malignant cells, for example as measured in a sample obtained from the subject, of at least 5%, 10%, 20%, 30%, 40%, 50%, 60%, 70%, 80%, 90% or 100% when compared to the amount or concentration of malignant cells before treatment. Methods of measuring the amount or concentration of malignant cells include, for example, qRT-PCR, and quantification of hyperproliferative specific biomarkers in a sample obtained from the subject.

As used herein the term “subject” refers to an individual, e.g., a human, having or at risk of having a specified condition, disorder or symptom. The subject may be a patient i.e. a subject in need of treatment in accordance with the invention. The subject may have received treatment for the condition, disorder or symptom. Alternatively, the subject has not been treated prior to treatment in accordance with the present invention.

The compositions described herein can be administered to the subject by any conventional route, including injection or by gradual infusion over time. The administration may, for example, be by infusion or by intramuscular, intravascular, intracavity, intracerebral, intralesional, rectal, subcutaneous, intradermal, epidural, intrathecal, percutaneous administration.

The compositions described herein may be in any form suitable for the above modes of administration. For example, compositions comprising modified cells may in any form suitable for infusion. As further examples, suitable forms for parenteral injection (including, subcutaneous, intramuscular, intravascular or infusion) include a sterile solution, suspension or emulsion. Alternatively, the route of administration may be by direct injection into the target area, or by regional delivery or by local delivery. The identification of suitable dosages of the compositions of the invention is well within the routine capabilities of a person of skill in the art.

Advantageously, the compositions described herein may be formulated for use in T cell receptor (TCR) gene transfer, an approach that is rapid, reliable and capable of generating large quantities of T cells with specificity for the Bob1 antigenic peptide (e.g. the peptide shown in SEQ ID NO:5), regardless of the patient's pre-existing immune repertoire. Using TCR gene transfer, modified cells suitable for infusion may be generated within a few days.

The compositions described herein are for administration in an effective amount. An “effective amount” is an amount that alone, or together with further doses, produces the desired (therapeutic or non-therapeutic) response. The effective amount to be used will depend, for example, upon the therapeutic (or non-therapeutic) objectives, the route of administration, and the condition of the patient/subject. For example, the suitable dosage of the composition of the invention for a given patient/subject will be determined by the attending physician (or person administering the composition), taking into consideration various factors known to modify the action of the composition of the invention for example severity and type of haematological malignancy, body weight, sex, diet, time and route of administration, other medications and other relevant clinical factors. The dosages and schedules may be varied according to the particular condition, disorder or symptom the overall condition of the patient/subject. Effective dosages may be determined by either in vitro or in vivo methods.

The pharmaceutical compositions described herein are advantageously presented in unit dosage form.

### Methods of Generating Binding Proteins (e.g. TCRs)

A method of generating a binding protein that is capable of specifically binding to a peptide containing a Bob1 antigen and does not bind to a peptide that does not contain the Bob1 antigen is also provided, comprising contacting a nucleic acid composition (or vector system) described herein with a cell under conditions in which the nucleic acid composition is incorporated and expressed by the cell.

In the context of the binding proteins described herein, the Bob1 antigen comprises or consists of the sequence of SEQ ID NO:5, or a functional fragment or variant thereof.

The method may be carried out on the (host) cell ex vivo or in vitro. Alternatively, the method may be performed in vivo, wherein the nucleic acid composition (or vector system) is administered to the subject and is contacted with the cell in vivo, under conditions in which the nucleic acid sequence is incorporated and expressed by the cell to generate the binding protein. In one example, the method is nota method of treatment of the human or animal body. Appropriate in vivo, in vitro and ex vivo methods for contacting a nucleic acid sequence (or vector systems) with a cell under conditions in which the nucleic acid sequence (or vector) is incorporated and expressed by the cell are well known, as described elsewhere herein.

As stated elsewhere herein, the binding protein comprise a TCR, an antigen binding fragment of a TCR, or a chimeric antigen receptor (CAR). Further details are provided elsewhere herein.

### General Definitions

As used herein “nucleic acid sequence”, “polynucleotide”, “nucleic acid” and “nucleic acid molecule” are used interchangeably to refer to an oligonucleotide sequence or polynucleotide sequence. The nucleotide sequence may be of genomic, synthetic or recombinant origin, and may be double-stranded or single-stranded (representing the sense or antisense strand). The term “nucleotide sequence” includes genomic DNA, cDNA, synthetic DNA, and RNA (e.g. mRNA) and analogs of the DNA or RNA generated, e.g., by the use of nucleotide analogs.

As used herein, “isolated nucleic acid sequence” or “isolated nucleic acid composition” refers to a nucleic acid sequence that is not in its natural environment when it is linked to its naturally associated sequence(s) that is/are also in its/their natural environment. In other words, an isolated nucleic acid sequence/composition is not a native nucleotide sequence/composition, wherein “native nucleotide sequence/composition” means an entire nucleotide sequence that is in its native environment and when operatively linked to an entire promoter with which it is naturally associated, which promoter is also in its native environment. Such a nucleic acid could be part of a vector and/or such nucleic acid or polypeptide could be part of a composition {e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region (“leader and trailer”) as well as intervening sequences (introns) between individual coding segments (exons).

As used herein “specifically binds” or “specific for” refers to an association or union of a binding protein (e.g., TCR receptor) or a binding domain (or fusion protein thereof) to a target molecule with an affinity or Kₐ (i.e., an equilibrium association constant of a particular binding interaction with units of 1/M) equal to or greater than 10⁵ M⁻¹ (which equals the ratio of the on-rate [kₒₙ] to the off-rate [k_{off}] for this association reaction), while not significantly associating or uniting with any other molecules or components in a sample. Binding proteins or binding domains (or fusion proteins thereof) may be classified as “high affinity” binding proteins or binding domains (or fusion proteins thereof) or as “low affinity” binding proteins or binding domains (or fusion proteins thereof). “High affinity” binding proteins or binding domains refer to those binding proteins or binding domains having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. Low affinity” binding proteins or binding domains refer to those binding proteins or binding domains having a Kₐ of up to 10⁷ M⁻¹, up to 10⁶ M⁻¹, up to 10⁵ M⁻¹. Alternatively, affinity can be defined as an equilibrium dissociation constant (K_{d}) of a particular binding interaction with units of M (e.g., 10⁻⁵ M to 10⁻¹³ M).

In certain embodiments, a receptor or binding domain may have “enhanced affinity,” which refers to selected or engineered receptors or binding domains with stronger binding to a target antigen than a wild type (or parent) binding domain. For example, enhanced affinity may be due to a Kₐ (equilibrium association constant) for the target antigen that is higher than the wild type binding domain, due to a K_{d} (dissociation constant) for the target antigen that is less than that of the wild type binding domain, due to an off-rate (k_{off}) for the target antigen that is less than that of the wild type binding domain, or a combination thereof. In certain embodiments, enhanced affinity TCRs can be codon optimized to enhance expression in a particular host cell, such as a cell of the immune system, a inducible pluripotent stem cell (iPSC), a hematopoietic stem cell, a T cell, a primary T cell, a T cell line, a NK cell, or a natural killer T cell (Scholten et al, Clin. Immunol. 119: 135, 2006). The T cell can be a CD4+ or a CD8+ T cell, or gamma-delta T cell.

As used herein, the term “Bob1 antigen” or “Bob1 peptide antigen” or “Bob1-containing peptide antigen” refers to a naturally or synthetically produced peptide portion of a Bob1 protein ranging in length from about 7 amino acids, about 8 amino acids, about 9 amino acids, about 10 amino acids, up to about 20 amino acids, which can form a complex with a MHC (e.g., HLA) molecule, and a binding protein of this disclosure specific for a Bob1 peptide:MHC (e.g., HLA) complex can specifically bind to such as complex. Typically, for the purposes of this disclosure, the Bob1 peptide antigen comprises or consists of the sequence of SEQ ID NO:5 and the Bob1 peptide antigen:HLA complex comprises SEQ ID NO:5:HLA*B35:01).

The term “ Bob1-specific binding protein,” as used herein, refers to a protein or polypeptide, such as a TCR or CAR, that specifically binds to a Bob1 peptide antigen (or to a Bob1 peptide antigen:HLA complex, e.g., on a cell surface), and does not bind a peptide sequence that does not include the Bob1 peptide antigen. Typically, for the purposes of this disclosure, the Bob1 peptide antigen comprises or consists of the sequence of SEQ ID NO:5 and the Bob1 peptide antigen:HLA complex comprises SEQ ID NO:5:HLA*B35:01).

In certain embodiments, a Bob1-specific binding protein specifically binds to a Bob1 peptide antigen (or a Bob1 peptide antigen:HLA complex) with a Kd of less than about 10⁻⁸ M, less than about 10⁻⁹ M, less than about 10⁻¹⁰ M, less than about 10⁻¹¹ M, less than about 10⁻¹² M, or less than about 10⁻¹³ M, or with an affinity that is about the same as, at least about the same as, or is greater than at or about the affinity exhibited by an exemplary Bob1-specific binding protein provided herein, such as any of the Bob1-specific TCRs provided herein, for example, as measured by the same assay. In certain embodiments, a Bob1-specific binding protein comprises a Bob1-specific immunoglobulin superfamily binding protein or binding portion thereof. Typically, for the purposes of this disclosure, the Bob1 peptide antigen comprises or consists of the sequence of SEQ ID NO:5 and the Bob1 peptide antigen:HLA complex comprises SEQ ID NO:5:HLA*B35:01).

The selective binding may be in the context of Bob1 antigen presentation by H LA-B*35:01. In other words, in certain embodiments, a binding protein that “specifically binds to a Bob1 antigen” may only do so when it is being presented (i.e. it is bound by) HLA-B*35:01 or is in an equivalent structural formation as when it is being presented by HLA-B*35:01.

By “specifically bind(s) to” as it relates to a T cell receptor, or as it refers to a recombinant T cell receptor, nucleic acid fragment, variant, or analog, or a modified cell, such as, for example, the Bob1 T cell receptors, and Bob1-expressing modified cells herein, is meant that the T cell receptor, or fragment thereof, recognizes, or binds selectively to the Bob 1 antigen (e.g. the Bob1 peptide LPHQPLATY). Under certain conditions, for example, in an immunoassay, for example an immunoassay discussed herein, the T cell receptor binds to Bob1 (e.g. the Bob1 peptide LPHQPLATY) and does not bind in a significant amount to other polypeptides. Thus the T cell receptor may bind to Bob1 (e.g. the Bob1 peptide LPHQPLATY) with at least 10, 100, or 1000, fold more affinity than to a control antigenic polypeptide. This binding may also be determined indirectly in the context of a modified T cell that expresses a Bob1 TCR. In assays such as, for example, an assay discussed herein, the modified T cell is specifically reactive against a multiple myeloma cell line and at least one malignant B cell lines such as, for example, ALL, CLL and mantle cell lymphoma cell lines. Thus, the modified Bob1-expressing T cell binds to a multiple myeloma cell line or a malignant B cell line with at least 10, 100, or 1000, fold more reactivity when compared to its reactivity against a control cell line that is not a multiple myeloma cell line or a malignant B cell line.

A “non-essential” (or “non-critical”) amino acid residue is a residue that can be altered from the wild-type sequence of (e.g., the sequence identified by SEQ ID NO herein) without abolishing or, more preferably, without substantially altering a biological activity, whereas an “essential” (or “critical”) amino acid residue results in such a change. For example, amino acid residues that are conserved are predicted to be particularly non-amenable to alteration, except that amino acid residues within the hydrophobic core of domains can generally be replaced by other residues having approximately equivalent hydrophobicity without significantly altering activity.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential (or non-critical) amino acid residue in a protein is preferably replaced with another amino acid residue from the same side chain family. Alternatively, in another embodiment, mutations can be introduced randomly, and the resultant mutants can be screened for activity to identify mutants that retain activity.

Calculations of sequence homology or identity (the terms are used interchangeably herein) between sequences are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 75%, 80%, 82%, 84%, 85%, 86%, 87%, 88%, 89%, 90%, 91%, 92%, 93%, 94%, 95%, 96%, 97%, 98%, 99%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman et al. (1970) J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

Alternatively, the percent identity between two amino acid or nucleotide sequences can be determined using the algorithm of Meyers et al. (1989) CABIOS 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-410). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, gapped BLAST can be utilized as described in Altschul et al. (1997, Nucl. Acids Res. 25:3389-3402). When using BLAST and gapped BLAST programs, the default parameters of the respective programs (e.g., XBLAST and NBLAST) can be used. See <http://www.ncbi.nlm.nih.gov>.

The polypeptides and nucleic acid molecules described herein can have amino acid sequences or nucleic acid sequences sufficiently or substantially identical to the sequences identified by SEQ ID NO. The terms “sufficiently identical” or “substantially identical” are used herein to refer to a first amino acid or nucleotide sequence that contains a sufficient or minimum number of identical or equivalent (e.g. with a similar side chain) amino acid residues or nucleotides to a second amino acid or nucleotide sequence such that the first and second amino acid or nucleotide sequences have a common structural domain or common functional activity. In other words, amino acid sequences or nucleic acid sequences having one or several (e.g. two, three, four etc) amino acid or nucleic acid substitutions compared to the corresponding sequences identified by SEQ ID NO may be sufficiently or substantially identical to the sequences identified by SEQ ID NO (provided that they retain the requisite functionality). In such examples, the one or several (e.g. two, three, four etc) amino acid or nucleic acid substitutions may be conservative substitutions. For example, amino acid or nucleotide sequences that contain a common structural domain having at least about 60%, or 65% identity, likely 75% identity, more likely 85%, 90%, 91%, 92%, 93%, 94%, 95%, 96%, 97%, 98% or 99% identity are defined herein as sufficiently or substantially identical.

TCR sequences are defined according to IMGT. See the LeFranc references herein for further details i.e. [1] Lefranc M.-P. “Unique database numbering system for immunogenetic analysis” Immunology Today, 18: 509 (1997). [2] Lefranc M.-P. “The IMGT unique numbering for immunoglobulins, T cell Receptors and Ig-like domains” The immunologist, 7, 132-136 (1999). [3] Lefranc M.-P. et al. “IMGT unique numbering for immunoglobulin and Tcell receptor variable domains and Ig superfamily V-like domains” Dev. Comp. Immunol., 27, 55-77 (2003). [4] Lefranc M.-P. et al. “IMGT unique numbering for immunoglobulin and T cell receptor constant domains and Ig superfamily C-like domains” Dev. Comp. Immunol., 2005, 29, 185-203 PMID: 15572068.

As used herein, the term “ex vivo” refers to “outside” the body. The term “in vitro” can be used to encompass “ex vivo” components, compositions and methods.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### EXAMPLES

### Identification of Bob1 Antigen as Target for Treatment of B Cell Malignancies

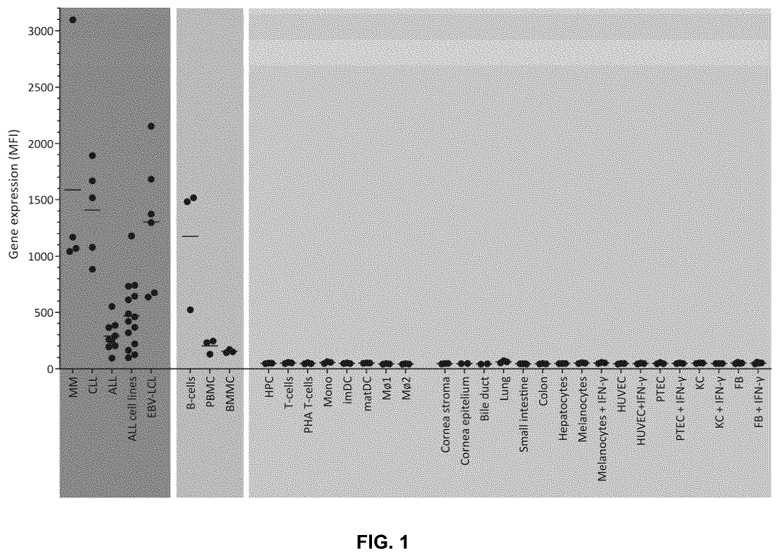

POU2AF1 is the gene encoding for the Bob1 protein. POU2AF1 was identified as a promising target for treatment of B cell malignancies based on previous microarray data generated by the inventors (1). POU2AF1 is expressed in acute lymphoblastic leukemia (ALL), chronic lymphocytic leukemia (CLL) and multiple myeloma (MM) (FIG. 1). Except for expression in healthy B cells, no expression in any other healthy tissues was detected.

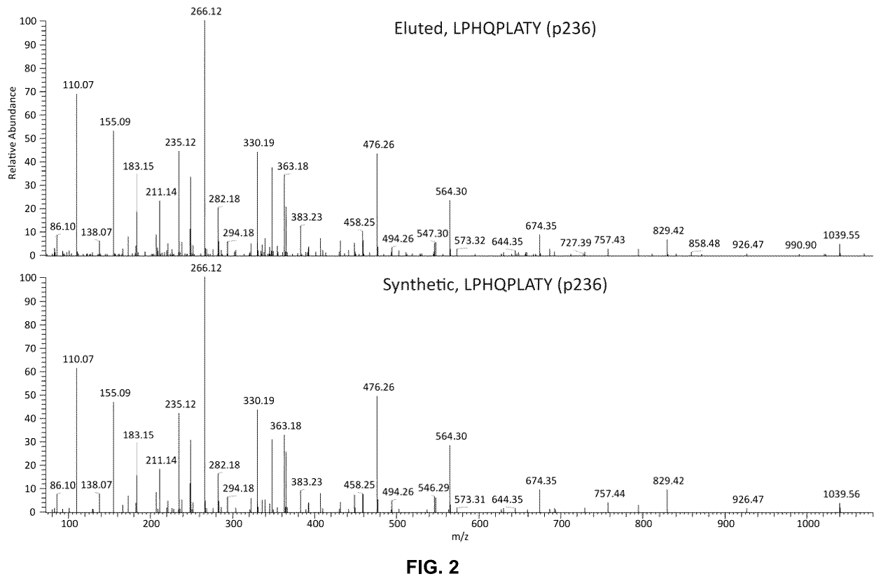

In order to target POU2AF1 expressing malignant B cells, potential TCR target peptides derived from the Bob1 protein which are processed and presented in HLA on the cell surface were determined. B cell malignancy material obtained from patients at moment of diagnosis was lysed and peptide-HLA complexes derived from the cell surface were isolated. Peptides were separated from HLA and peptide sequences were identified using mass spectrometry. This resulted in identification of five peptides derived from the Bob1 protein presented in frequently occurring HLA alleles HLA-A*02:01, HLA-B*07:02 and HLA-B*35:01 (table 2). Synthetic peptides were ordered and peptide sequences were confirmed by comparing mass spectra of synthetic peptides to spectra from eluted peptide (FIG. 2).

TABLE 2
Sequences of peptides eluted from B cell
malignancy material identified by mass
spectrometry, HLA alleles from which peptides are
derived with peptide numbers assigned for
reference.

| | | Assigned |
|---|---|---|
| | HLA | peptide |
| Peptide sequence | allele | number |
| YALNHTLSV (SEQ ID NO: 1) | A*02: 01 | p127 |
| APALPGPQF (SEQ ID NO: 2) | B*07: 02 | p113 |
| APAPTAVVL (SEQ ID NO: 3) | B*07: 02 | p114 |
| APARPYQGV (SEQ ID NO: 4) | B*07: 02 | p115 |
| APAPTAVVL (SEQ ID NO: 3) | B*35: 01 | p233 |
| LPHQPLATY (SEQ ID NO: 5) | B*35: 01 | p236 |

### Successful Isolation of Clinically Relevant Bob1 Targeting T Cells

In order to be of clinical relevance, TCRs must recognize target peptide with high affinity. In HLA-A*02:01, B*07:02 and B*35:01 expressing individuals, high affinity T cells recognizing Bob1 derived self-peptides are deleted during thymic selection to prevent autoimmune disease. In contrast, in target HLA negative individuals, high affinity T cells specific for self-peptides can be present in the T cell repertoire. Therefore, PBMCs from healthy donors not expressing target HLA alleles were used and incubated with peptide-HLA tetramers to isolate T cells. Tetramer bound CD8 positive T cells were single-cell sorted and clonally expanded. Functionality was assessed by cytokine production after overnight coculture with Bob1 antigen negative K562 cells loaded with target peptides. For two of the peptides (APAPTAVVL (SEQ ID NO:3) in HLA-B*07:02 and YALNHTLSV (SEQ ID NO:1) in HLA-A*02:01) specific TCRs were previously identified (2). In this study isolation of other T cell clones recognizing Bob1 peptide in HLA-A*02:01 or B*07:02 was unsuccessful. However, two HLA-B*35:01 restricted Bob1 specific T cell clones, clone 1C5.6 and clone 4H5.6, were identified. T cell clone 1C5.6 and T cell clone 4H5.6 recognized K562 cells transduced (Td) with HLA-B*35:01 loaded with Bob1 derived peptides p236 and p233 (FIG. 3a). Tetramer stain revealed specificity for p236: LPHQPLATY (SEQ ID NO:5) for both T cell clones, although the mean fluorescence intensity of the tetramer staining was higher for clone 1C5.6 compared to 4H5.6 (FIG. 3b). To gain insight in the potency of the identified T cell clone, recognition of endogenously processed and presented peptides was assessed by stimulation with K562 cells transduced with HLA-B*35:01 and the POU2AF1 gene encoding the Bob1 protein. Potent recognition of target gene Td target cells suggested high affinity for p236 for clone 1C5.6, which was confirmed in a peptide titration experiment where K562 cells loaded with decreased peptide concentrations were recognized when only 1 pg/ml of peptide LPHQPLATY (SEQ ID NO:5) was added, whereas clone 4H5.6 exhibited a much lower affinity (FIG. 3c). To assess clinical relevance of T cell clone 1C5.6 and T cell clone 4H5.6 in more detail, T cells were co-cultured with multiple Bob1 expressing ALL and MM derived cell lines. Potent effector cytokine production was observed upon stimulation with Bob1 expressing HLA-B*35:01 positive target cells while antigen negative or HLA-B*35:01 negative cells were not recognized by clone 1C5.6 (FIG. 3d). In agreement with the lower affinity of 4H5.6 for the Bob1 peptide, clone 4H5.6 only recognized 2 out of 5 Bob1 expressing ALL and MM derived cell lines, indicating that this clone is of too low affinity to proceed further analyses. Potent recognition of all 5 Bob1 expressing HLA-B*35:01 positive B cell malignancy cell lines revealed great promise for clinical application of the TCR from T cell clone 1C5.6.

In TCR gene therapy, treatment safety is equally important to potency to prevent life threatening toxicity. To determine cross reactivity with other HLA alleles, T cell clone 1C5.6 was stimulated with a panel of EBV-LCLs expressing all HLA-I alleles with a frequency >1% in the Caucasian population (FIG. 4a, table 3).

TABLE 3
HLA typing of EBV-LCLs used in this study

| EBV-LCL | HLA-A | HLA-B | HLA-C |
|---|---|---|---|
| GMK | 23:01:01-02:01 | 41:01-40:01 | 17:01:01:01-03:04:01:01 |
| RSB | 02:01-03:01/03:03/03:04 | 44:02-57:01 | 06:02-07:04/07:12/07:11 |
| EBK | 02:05-02:05 | 58:01-58:01 | unknown |
| ERC | 02:01-02:01 | 13:02-44:02 | 05:01-06:02 |
| URN | 02:01-03:01 | 08:01:01-50:01:01 | 06:02:01-07:01 |
| BBD | 02:01-02:05 | 15:01-45:01 | 01:02-06:02 |
| ABC | 02:01:01-11:01:01:01 | 44:05:01-51:01:01:01 | 02:02:02-14:02:01 |
| NMJ | 02:01-66:01/66:04 | 40:01/40:11/40:14-41:02 | 03:04/03:08/03:09-17 |
| QBO | 24:02:01:01-31:01:02 | 07:02/07:61-35:08:01 | 04:01-07:02 |
| JMQ | 02:01-24:02:01:01 | 35:02-44:02 | 04:01-05:01 |
| HRK | 03:01-25:01 | 15:17-18:01/18:03/18:05 | 07:01/07:05/07:06- |
| | | | 12:03/12:06 |
| MSV | 03:01-33:01 | 07:02-14:02 | 07:02-08:02 |
| JBX | 02:01-30:02 | 15:01-39:01 | 03:03-12:03 |
| IGU | 03:01-26:01 | 07:02:01-14:01 | 07:02-08:02 |
| LSR | 32:01-68:01 | 35:03-52:01 | 12:02-12:03 |
| HBM | 02:01:01-02:01:01 | 15:01:01:01-51:01:01 | 03:03:01-15:02:01 |
| JBZ | 01:01-02:01 | 07:02-18:01 | 07:01-07:02 |
| RKO | 02:05-29:02 | 27:05-44:03 | 01:02-16:01:01 |
| MSF | 03:01/03:03/03:04-30:01 | 07:02-38:01 | 07:02/07:03/07:05- |
| | | | 12:03/12:06 |
| BSR | 02:01-68:01 | 35:03-37:01 | 04:01-06:02 |
| UWI | 02:01-24:02 | 07:02:01-40:02:01 | 02:02:02-07:02:01 |
| ABF | 30:04-68:02 | 38:01-55:01 | 03:03-12:03 |
| GGT | 26:01/26:08/26:02- | 14:01-49:01 | 07:01/07:05/07:06- |
| | 31:01/31:02/31:06 | | 08:02/08:07 |
| AAJ | 03:01/03:03/03:04- | 40:02/40:35/40:37-56:01 | 01:02/01:06/01:07- |
| | 11:01/11:02/11:03 | | 02:02/02:04/02:08 |
| AKB | 01:01-02:01 | 37:01-39:01 | 06:02-07:02 |

In addition, cross reactivity with peptides presented in HLA-B*35:01 was investigated by stimulation with Bob1 negative cell lines from various origins Td with HLA-B*35:01 (FIG. 4b).

In both experiments no cross reactivities were observed while positive control cells were potently recognized indicating that the TCR of clone 1C5.6 could safely be used in the clinic.

### CD8 T Cells Induce Potent Lysis of Patient Derived B Cell Malignancy Samples Upon Introduction of TCR 1C5.6

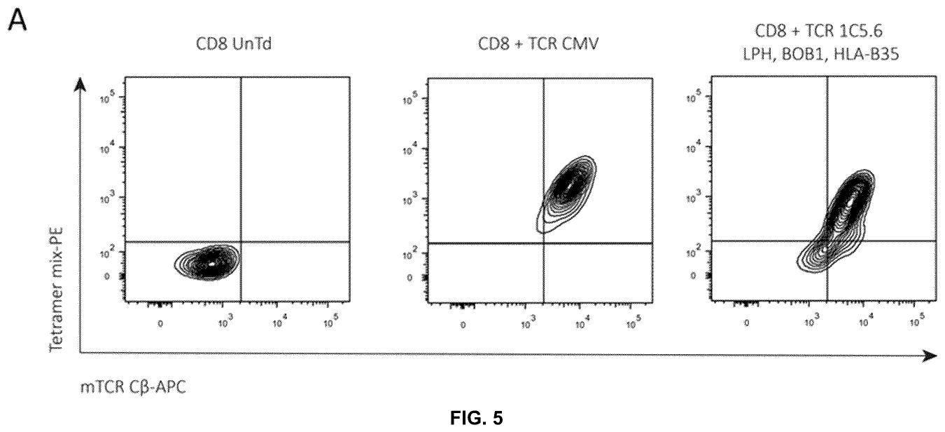

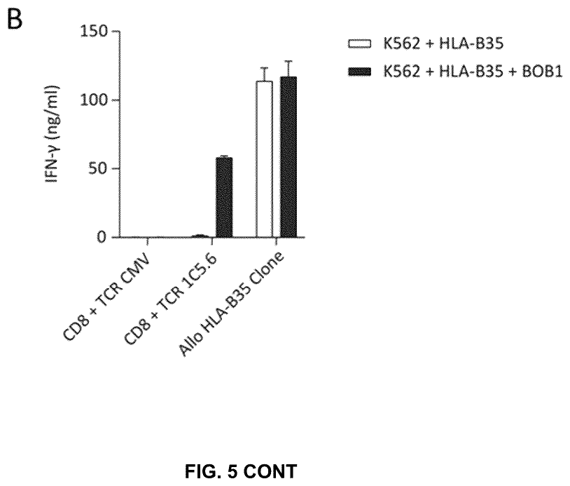

The efficacy and safety profile of T cell clone 1C5.6 makes the TCR of clone 1C5.6 an excellent candidate for further development for TCR gene therapy of B cell malignancies. The TCR sequence of T cell clone 1C5.6 was successfully identified. Upon retroviral transfer of TCR 1C5.6 in CD8 T cells, Bob1 specific recognition was demonstrated by tetramer stain and cytokine production after stimulation with Bob1 antigen expressing K562 cells (FIG. 5).

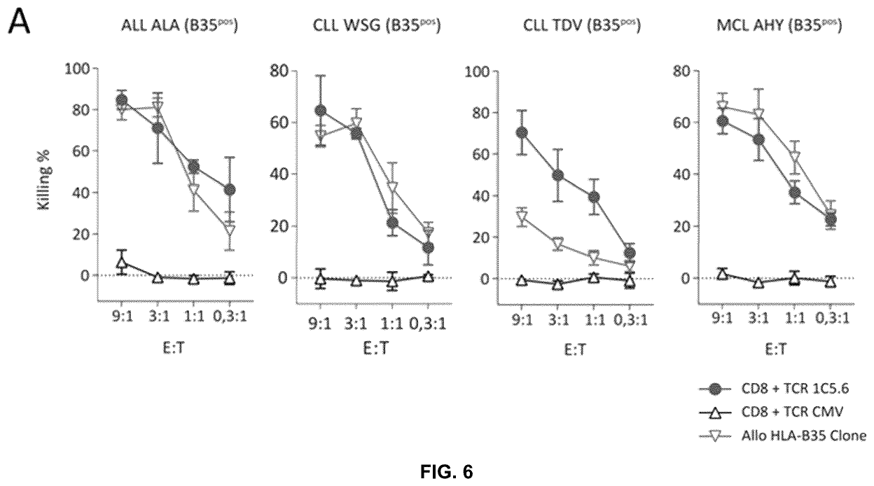

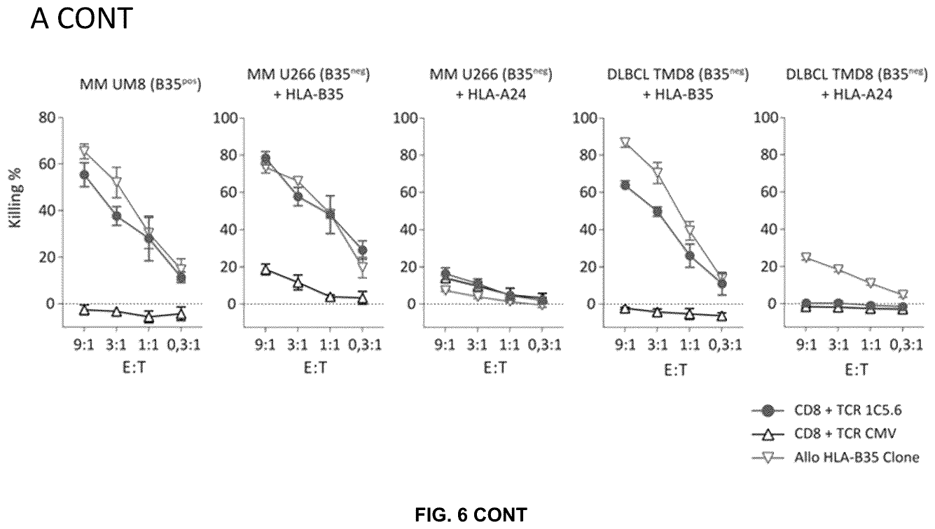

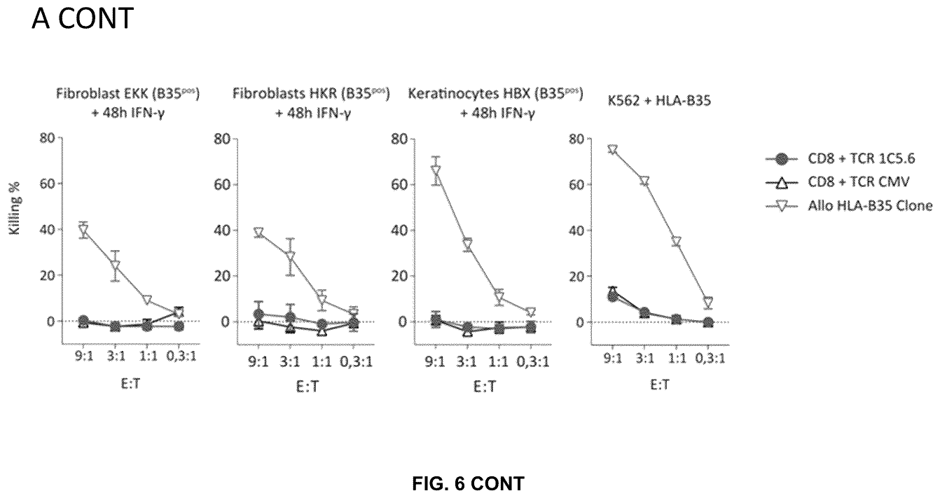

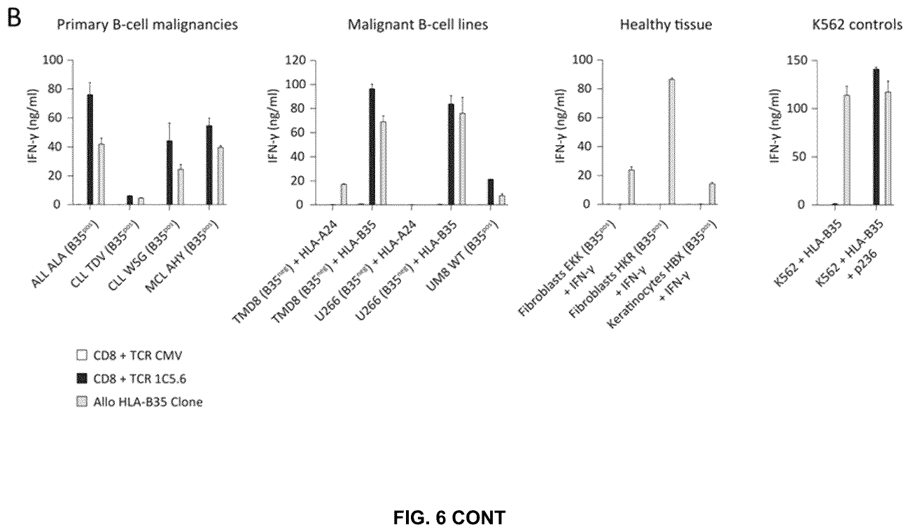

TCR 1C5.6 Td T cells, but not control TCR T cells induced potent lysis of patient derived ALL, CLL and mantle cell lymphoma (MCL) samples as well as MM and diffuse larger B cell lymphoma (DLBCL) cell lines expressing HLA-B*35:01 (FIG. 6a). In absence of target HLA, no lysis of MM cell line UM9 and DLBCL cell line TMD8 was observed. In addition, Bob1 negative HLA-B*35:01 positive healthy tissues were not lysed, confirming the previously observed safety of this TCR. Positive control allo HLA-B*35:01 T cell clone lysed all HLA-B*35:01 positive target cells, confirming HLA-B*35:01 expression and stimulatory capacity. Lysis by TCR 1C5.6 Td T cells and allo HLA-B*35:01 T cell clone was accompanied by effector cytokine production, no cytokine was produced when target cell lysis was absent (FIG. 6b). In summary, T cell clone 1C5.6 is a high affinity T cell clone recognizing peptide LPHQPLATY (SEQ ID NO:5) derived from the Bob1 protein presented in HLA-B*35:01. The recognition profile of T cell clone 1C5.6 is highly restricted to Bob1 antigen expressing HLA-B*35:01 positive target cells. Upon sequencing and transfer of TCR 1C5.6 T, cells induced potent lysis of a broad range of primary B cell malignancies and B cell lines while Bob1 antigen negative cells were not lysed. To conclude, the inventors have demonstrated that the identified TCR, TCR 1C5.6 is safe and effective and therefore promising for TCR gene therapy of B cell malignancies.

### Potent In Vivo Anti-Tumor Efficacy of BOB1 TCR Td CD8 T Cells

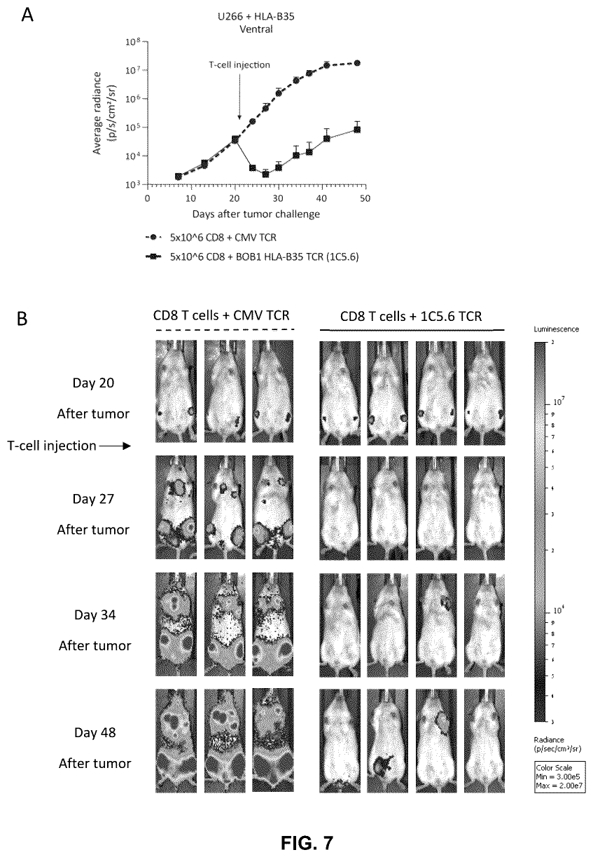

The inventors investigated the in vivo killing capacity of TCR 1C5.6 (BOB1 HLA-B35) Td CD8 T cells in a previously established xenograft model for treatment of established multiple myeloma. NSG mice were inoculated with BOB1 expressing, HLA-B35 transduced multiple myeloma cell line U266. Upon treatment with BOB1 HLA-B35 restricted TCR 1C5.6 Td CD8 T cells a strong anti-tumor effect was observed (FIG. 7). Tumors in TCR 1C5.6 treated mice reached their minimal size 6 days after T-cell infusion, when the mean tumor burden was 148-fold lower in 1C5.6 TCR treated mice compared to control TCR treated mice. Despite near complete tumor eradication, U266 regrows after day 6 post T cells likely due to absence of the required human cytokine environment.

### Materials and Methods

For further details on the methodology used see WO2016/071758, which in incorporated herein by reference in its entirety.

### Generation of Peptide-HLA Tetramers

Synthetic peptides were generated in house using standard Fmoc chemistry. Recombinant HLA-A*01:01, A*24:02, B*08:01, B*35:01 heavy chains and human B2M were produced in house in Escherichia coli. Peptide, heavy chain and B2M were combined to fold pHLA monomers. pHLA monomers were biotinylated and purified by gel filtration using high-performance liquid chromatography. PE labelled pHLA-tetramers were generated by mixing biotinylated monomers with PE conjugated streptavidin (Invitrogen, Thermo Fischer Scientific), in the optimal monomer:streptavidin ratio. pMHC tetramers were stored at 4° C. for short term storage and at −80° C. for long term storage.

### T Cell Isolation and Culture

Buffy coats were obtained from healthy donors negative for HLA-A1, HLA-A24, HLA-B8 and HLA-B*35 after informed consent (Sanquin). PBMCs were isolated using Ficoll gradient separation and incubated with pHLA-tetramers for 1 hour at 4° C. Cells were washed and pHLA-tetramer bound cells were enriched by magnetic associated cell sorting (MACS) using anti-PE beads (Miltenyi Biotec). The positive fraction was stained with CD8-Alexa fluor 700 (Invitrogen/Catlag) and FITC labelled CD4, CD14 and CD19 (BD pharmingen). pHLA-tetramer⁺, CD8⁺ cells were single cell sorted using an Aria III cell sorter (BD Biosciences) in a 96 well round bottom plate containing 5×10\hat{ }4 irradiated PBMCs (35Gy) and 5×10\hat{ }3 EBV-JY cells (50Gy) in 100 ul T cell medium (TCM) with 0.8 μg/ml phytohemagglutinin (PHA; Oxoid Microbiology Products, Thermo Fischer Scientific). TCM contains IMDM (Lonza), 1% Penicillin/Streptomycin (Pen/Strep; Lonza), 1.5% glutamine (Lonza), 100 IU/ml IL-2 (Proleukin; Novartis Pharma), 5% fetal bovine serum (FBS; Gibco, Life Technologies) and 5% human serum. T cell clones were restimulated every 10-15 days with irradiated feeder cells and PHA or cryopreserved until further use.

### Target Cell Culture and Generation of Transduced Cells

Cell lines were cultured in IMDM (Lonza), 1% Pen/Strep (Lonza), 1.5% Glutamine (Lonza) and 10% FBS (Gibco, Life Technologies). Primary malignant samples were defrosted and rested overnight at 37° C. in medium containing 10% human serum before use in experiments. HLA and target gene transduced (Td) target cells were generated by retroviral transduction with HLA alone or with target gene and HLA combined. Candidate genes and HLA alleles were expressed in MP71 retroviral backbone vectors with marker genes truncated nerve growth factor receptor (NGF-R), CD34 or mouseCD19. Transduced cells were MACS or FACS enriched for marker gene and/or HLA-I expression using HLA-ABC FITC (serotec), NGF-R PE (BD/Pharmingen), mCD19 PE (BD) or CD34 (fluorochroom, leverancier).

### T Cell Recognition Assay

Target cell recognition was determined by incubating 5,000 T cells, all experiments except for the first peptide recognition screen, with target cells in a Effector:Target (E:T) 1:6 ratio in a 384 well tissue culture plate. To compensated for the difference in cell size primary samples were tested in E:T 1:12 or 1:20. T cells were washed twice before use in experiments to remove expansion-related cytokines. After overnight (O/N) incubation recognition was determined by measuring IFN-γ and/or GM-CSF production in supernatants by ELISA (Sanquin and R&D systems). Peptide loaded target cells were loaded with 100 nM per peptide or decreasing peptide concentrations starting at 1 μM for peptide titration experiments. In the first peptide recognition screening T cells were not counted, per clone 100 ul was used and divided between four targets, therefore T cell numbers varied between T cell clones as a result of differences in expansion. T cell mediated cytotoxicity was measured using ⁵¹Cr-release experiments. Target cells were incubated 1 hour at 37° C. with 100 μCi Na₂⁵¹CrO₄. Target cells were washed and co-cultured with T cells at various E:T ratios for 6 hours in 96-well U-bottom culture plates. Supernatants were harvested and transferred to 96-well LumaPlates (Perkin Elmer). Spontaneous and maximum ⁵¹Cr-release was determined using TCM alone or TCM containing 1% Triton-X 100 (Sigma-Aldrich), respectively. ⁵¹Cr-release was measured in counts per minute (CPM) using a 2450 Microbeta² plate counter (PerkinElmer). Percentage target cell killing was calculated using % killing=((CPMₜₑₛₜ−averageCPMₛₚₒₙ)/(averageCPMₘₐₓ−averageCPMₛₚₒₙ))*100.

### Quantitative RT-PCR

Total RNA was isolated from 0.5−5×10\hat{ }6 cells using the Small Scale Kit or ReliaPrep RNA cell mini prep system according to manufacturer's protocol (Ambion, Promega respectively). Total RNA was converted to cDNA using Moloney murine leukemia virus reverse transcriptase and Oligo (dT) primer (Invitrogen). qRT-PCR was performed using Fast Start TagDNA Polymerase (Roche) and EvaGreen (Biotum), gene expression was measured on the Lightcycler 480 (Roche).

### TCR Identification

To identify TCRα and TCRβ sequences of T cell clones, mRNA was isolated from 1×10\hat{ }6 cells using the mRNA DIRECT kit (Invitrogen). Barcoded TCR cDNA was generated in two rounds of PCR. In the first round TCR cDNA was generated using reverse primers in the TCR constant alfa and beta regions, SMARTScribe Reverse Transcriptase (Takara, Clontech) and a template switching oligo forward primer. In the second round of PCR a 5′ illumina adapter and a barcode sequence was included that allows discrimination between TCRs of different T cell clones. cDNA concentrations were measured by Qbit, comparable amounts of cDNA of different T cell clones were pooled. TCR sequences were identified by HiSeq (genome scan). Hiseq data was analysed using MiXCR and ImMunoGeneTics (IMGT) database to determine the Vα /Vβ family. V(D)J segments of the TCRα and TCRβ were codon optimized and cloned into the modified MP71-TCR-flex retroviral vector. To increase expression and preferential pairing of the introduced TCRαβ chain, the MP71-TCR-flex vector contains codon-optimized and cysteine-modified murine TCRαβ constant domains and P2A sequence to link TCR chains. Phoenix-AMPHO cells were transfected, after 48 and 72 hours virus supernatant was harvested and stored at −80° C.

### TCR Transfer to Donor T Cells

CD8⁺ T cells were isolated from healthy donor PBMCs by MACS using anti-CD8 microbeads (Miltenyi Biotec). CD8⁺ T cells were activated with irradiated autologous PBMCs (35Gy) and 0.8 μg/ml PHA. On day 2, retroviral supernatants were added to 24-well suspension culture plates (Greiner Bio-One) precoated with 30 mg/mL retronectin (Takara) and blocked with 2% human serum albumin (Sanquin). Plates were spun down for 20 min, 2000 g at 4° C. Virus supernatant was removed and 0.3×10\hat{ }6 activated T cells were transferred to each well. After O/N incubation T cells were transferred to a 24-well culture plate (Costar). On day 7 after T cell activation TCR Td T cells were MACS enriched using anti-mouse TCR-Cβ (mTCR) APC antibody (BD Pharmingen) followed by anti-APC MicroBeads (Miltenyi Biotec) according to manufacturer's protocol. TCR Td T cells were functionally tested between day 10-12 after activation. For the safety screening of TCR 6B10.12, endogenous TCRαβ knock out (KO) of healthy donor CD8 T cells was performed prior to TCR Td as described by Morton et al. 2020.

To assess TCR expression and tetramer binding cells were stained using mTCR APC antibody and PE pHLA-tetramers. Cells were measured on the LSR II (BD Bioscience) and data was analysed with Flowjo software.

### Nucleic Acid and Amino Acid Sequences of Interest

SEQ ID NO: 1 (Bob1 peptide): YALNHTLSV
SEQ ID NO: 2 (Bob1 peptide): APALPGPQF
SEQ ID NO: 3 (Bob1 peptide): APAPTAVVL
SEQ ID NO: 4 (Bob1 peptide): APARPYQGV
SEQ ID NO: 5 (Bob1 peptide): LPHQPLATY
SEQ ID NO: 6 (amino acid sequence for CDR1 of Vα domain of TCR 1C5.6): SSVSVY
SEQ ID NO: 7 (nucleic acid sequence for CDR1 of Vα domain of TCR 1C5.6):
TCGTCTGTTTCAGTGTAT
SEQ ID NO: 8 (codon optimized nucleic acid sequence for CDR1 of Vα domain of TCR 1C5.6):
AGCAGCGTGAGCGTGTAC
SEQ ID NO: 9 (amino acid sequence for CDR2 of Vα domain of TCR 1C5.6): YLSGSTLV
SEQ ID NO: 10 (nucleic acid sequence for CDR2 of Vα domain of TCR 1C5.6):
TATTTATCAGGATCCACCCTGGTT
SEQ ID NO: 11 (codon optimized nucleic acid sequence for CDR2 of Vα domain of TCR
1C5.6): TACCTGAGCGGGAGCACACTGGTG
SEQ ID NO: 12 (amino acid sequence for CDR3 of Vα domain of TCR 1C5.6):
CAVKVSNAGGTSYGKLTF
SEQ ID NO: 13 (nucleic acid sequence for CDR3 of Vα domain of TCR 1C5.6):
TGTGCTGTGAAGGTGTCTAACGCTGGTGGTACTAGCTATGGAAAGCTGACATTT
SEQ ID NO: 14 (codon optimized nucleic acid sequence for CDR3 of Vα domain of TCR
1C5.6):
TGCGCCGTGAAGGTTAGTAACGCCGGCGGCACTAGCTACGGAAAGTTGACCTTC
SEQ ID NO: 15 (amino acid sequence for CDR1 of Vβ domain of TCR 1C5.6): LNHDA
SEQ ID NO: 16 (nucleic acid sequence for CDR1 of Vβ domain of TCR 1C5.6):
TTGAACCACGATGCC
SEQ ID NO: 17 (codon optimized nucleic acid sequence for CDR1 of Vβ domain of TCR
1C5.6): CTGAACCACGATGCC
SEQ ID NO: 18 (amino acid sequence for CDR2 of Vβ domain of TCR 1C5.6): SQIVND
SEQ ID NO: 19 (nucleic acid sequence for CDR2 of Vβ domain of TCR 1C5.6):
TCACAGATAGTAAATGAC
SEQ ID NO: 20 (codon optimized nucleic acid sequence for CDR2 of Vβ domain of TCR
1C5.6): AGTCAGATTGTGAACGAT
SEQ ID NO: 21 (amino acid sequence for CDR3 of Vβ domain of TCR 1C5.6):
CASSIAQGADTQYF
SEQ ID NO: 22 (nucleic acid sequence for CDR3 of Vβ domain of TCR 1C5.6):
TGTGCCAGTAGTATTGCTCAGGGTGCAGATACGCAGTATTTT
SEQ ID NO: 23 (codon optimized nucleic acid sequence for CDR3 of Vβ domain of TCR
1C5.6): TGCGCTAGCAGCATTGCTCAGGGCGCTGATACACAGTACTTT
SEQ ID NO: 24 (amino acid sequence for Vα (VJ) domain of TCR 1C5.6):
MLLLLVPAFQVIFTLGGTRAQSVTQLDSQVPVFEEAPVELRCNYSSSVSVYLFWYVQYPNQ
GLQLLLKYLSGSTLVESINGFEAEFNKSQTSFHLRKPSVHISDTAEYFCAVKVSNAGGTSYG
KLTFGQGTILTVHP
SEQ ID NO: 25 (nucleic acid sequence for Vα (VJ) domain of TCR 1C5.6):
ATGCTCCTGCTGCTCGTCCCAGCGTTCCAGGTGATTTTTACCCTGGGAGGAACCAGAG
CCCAGTCTGTGACCCAGCTTGACAGCCAAGTCCCTGTCTTTGAAGAAGCCCCTGTGGA
GCTGAGGTGCAACTACTCATCGTCTGTTTCAGTGTATCTCTTCTGGTATGTGCAATACC
CCAACCAAGGACTCCAGCTTCTCCTGAAGTATTTATCAGGATCCACCCTGGTTGAAAGC
ATCAACGGTTTTGAGGCTGAATTTAACAAGAGTCAAACTTCCTTCCACTTGAGGAAACC
CTCAGTCCATATAAGCGACACGGCTGAGTACTTCTGTGCTGTGAAGGTGTCTAACGCTG
GTGGTACTAGCTATGGAAAGCTGACATTTGGACAAGGGACCATCTTGACTGTCCATCCA
SEQ ID NO: 26 (codon optimized nucleic acid sequence for Vα (VJ) domain of TCR 1C5.6):
ATGCTGCTGCTGCTGGTGCCCGCCTTCCAGGTGATCTTCACCCTGGGCGGCACCCGG
GCCCAGAGCGTGACACAGCTGGATAGCCAGGTGCCCGTGTTCGAGGAGGCCCCCGTG
GAGCTGCGGTGCAACTACAGCAGCAGCGTGAGCGTGTACCTGTTCTGGTACGTGCAGT
ACCCCAACCAGGGACTGCAGCTGCTGCTGAAGTACCTGAGCGGGAGCACACTGGTGG
AGAGCATTAACGGGTTTGAAGCTGAGTTCAACAAATCCCAGACATCTTTTCACCTGAGG
AAGCCAAGCGTGCACATTTCCGACACCGCCGAGTACTTCTGCGCCGTGAAGGTTAGTA
ACGCCGGCGGCACTAGCTACGGAAAGTTGACCTTCGGACAGGGGACAATCCTGACTGT
CCATCCC
SEQ ID NO: 27 (amino acid sequence for Vβ (VDJ) domain of TCR 1C5.6):
MSNQVLCCVVLCFLGANTVDGGITQSPKYLFRKEGQNVTLSCEQNLNHDAMYWYRQDPG
QGLRLIYYSQIVNDFQKGDIAEGYSVSREKKESFPLTVTSAQKNPTAFYLCASSIAQGADTQ
YFGPGTRLTVL
SEQ ID NO: 28 (nucleic acid sequence for Vβ (VDJ) domain of TCR 1C5.6):
ATGAGCAACCAGGTGCTCTGCTGTGTGGTCCTTTGTTTCCTGGGAGCAAACACCGTGG
ATGGTGGAATCACTCAGTCCCCAAAGTACCTGTTCAGAAAGGAAGGACAGAATGTGAC
CCTGAGTTGTGAACAGAATTTGAACCACGATGCCATGTACTGGTACCGACAGGACCCA
GGGCAAGGGCTGAGATTGATCTACTACTCACAGATAGTAAATGACTTTCAGAAAGGAGA
TATAGCTGAAGGGTACAGCGTCTCTCGGGAGAAGAAGGAATCCTTTCCTCTCACTGTGA
CATCGGCCCAAAAGAACCCGACAGCTTTCTATCTCTGTGCCAGTAGTATTGCTCAGGGT
GCAGATACGCAGTATTTTGGCCCAGGCACCCGGCTGACAGTGCTC
SEQ ID NO: 29 (codon optimized nucleic acid sequence for Vβ (VDJ) domain of TCR 1C5.6):
ATGAGCAACCAGGTGCTGTGCTGCGTGGTGCTGTGCTTTCTTGGCGCTAACACAGTGG
ATGGAGGCATTACACAGAGCCCAAAGTACCTGTTTAGAAAGGAGGGGCAGAACGTGAC
ACTGAGCTGTGAGCAGAACCTGAACCACGATGCCATGTACTGGTACAGACAAGATCCA
GGACAGGGGCTGAGACTGATCTACTACAGTCAGATTGTGAACGATTTTCAGAAGGGAG
ATATTGCCGAGGGCTACAGCGTGTCTAGGGAGAAGAAGGAGTCTTTTCCACTGACAGT
GACTTCAGCCCAGAAGAACCCTACAGCCTTTTACCTGTGCGCTAGCAGCATTGCTCAG
GGCGCTGATACACAGTACTTTGGACCTGGGACAAGGCTGACAGTGCTG
SEQ ID NO: 30 (amino acid sequence for Vα (VJ) domain and constant domain of TCR 1C5.6):
MLLLLVPAFQVIFTLGGTRAQSVTQLDSQVPVFEEAPVELRCNYSSSVSVYLFWYVQYPNQ
GLQLLLKYLSGSTLVESINGFEAEFNKSQTSFHLRKPSVHISDTAEYFCAVKVSNAGGTSYG
KLTFGQGTILTVHPNIQNPDPAVYQLRDSKSSDKSVCLFTDFDSQTNVSQSKDSDVYITDKT
VLDMRSMDFKSNSAVAWSNKSDFACANAFNNSIIPEDTFFPSPESSCDVKLVEKSFETDTN
LNFQNLSVIGFRILLLKVAGFNLLMTLRLWSS
SEQ ID NO: 31 (amino acid sequence for Vα (VJ) domain of TCR 1C5.6 and constant domain
(murine)):
MLLLLVPAFQVIFTLGGTRAQSVTQLDSQVPVFEEAPVELRCNYSSSVSVYLFWYVQYPNQ
GLQLLLKYLSGSTLVESINGFEAEFNKSQTSFHLRKPSVHISDTAEYFCAVKVSNAGGTSYG
KLTFGQGTILTVHPDIQNPEPAVYQLKDPRSQDSTLCLFTDFDSQINVPKTMESGTFITDKCV
LDMKAMDSKSNGAIAWSNQTSFTCQDIFKETNATYPSSDVPCDATLTEKSFETDMNLNFQN
LSVMGLRILLLKVAGFNLLMTLRLWSS
SEQ ID NO: 32 (nucleic acid sequence for Vα (VJ) domain and constant domain of TCR
1C5.6):
ATGCTCCTGCTGCTCGTCCCAGCGTTCCAGGTGATTTTTACCCTGGGAGGAACCAGAG
CCCAGTCTGTGACCCAGCTTGACAGCCAAGTCCCTGTCTTTGAAGAAGCCCCTGTGGA
GCTGAGGTGCAACTACTCATCGTCTGTTTCAGTGTATCTCTTCTGGTATGTGCAATACC
CCAACCAAGGACTCCAGCTTCTCCTGAAGTATTTATCAGGATCCACCCTGGTTGAAAGC
ATCAACGGTTTTGAGGCTGAATTTAACAAGAGTCAAACTTCCTTCCACTTGAGGAAACC
CTCAGTCCATATAAGCGACACGGCTGAGTACTTCTGTGCTGTGAAGGTGTCTAACGCTG
GTGGTACTAGCTATGGAAAGCTGACATTTGGACAAGGGACCATCTTGACTGTCCATCCA
AATATCCAGAACCCTGACCCTGCCGTGTACCAGCTGAGAGACTCTAAATCCAGTGACAA
GTCTGTCTGCCTATTCACCGATTTTGATTCTCAAACAAATGTGTCACAAAGTAAGGATTC
TGATGTGTATATCACAGACAAAACTGTGCTAGACATGAGGTCTATGGACTTCAAGAGCA
ACAGTGCTGTGGCCTGGAGCAACAAATCTGACTTTGCATGTGCAAACGCCTTCAACAAC
AGCATTATTCCAGAAGACACCTTCTTCCCCAGCCCAGAAAGTTCCTGTGATGTCAAGCT
GGTCGAGAAAAGCTTTGAAACAGATACGAACCTAAACTTTCAAAACCTGTCAGTGATTG
GGTTCCGAATCCTCCTCCTGAAAGTGGCCGGGTTTAATCTGCTCATGACGCTGCGGTT
GTGGTCCAGCTGA
SEQ ID NO: 33 (codon optimized nucleic acid sequence for Vα (VJ) domain of TCR 1C5.6 and
constant domain (murine)):
ATGCTGCTGCTGCTGGTGCCCGCCTTCCAGGTGATCTTCACCCTGGGCGGCACCCGG
GCCCAGAGCGTGACACAGCTGGATAGCCAGGTGCCCGTGTTCGAGGAGGCCCCCGTG
GAGCTGCGGTGCAACTACAGCAGCAGCGTGAGCGTGTACCTGTTCTGGTACGTGCAGT
ACCCCAACCAGGGACTGCAGCTGCTGCTGAAGTACCTGAGCGGGAGCACACTGGTGG
AGAGCATTAACGGGTTTGAAGCTGAGTTCAACAAATCCCAGACATCTTTTCACCTGAGG
AAGCCAAGCGTGCACATTTCCGACACCGCCGAGTACTTCTGCGCCGTGAAGGTTAGTA
ACGCCGGCGGCACTAGCTACGGAAAGTTGACCTTCGGACAGGGGACAATCCTGACTGT
CCATCCCGACATTCAGAACCCGGAACCGGCTGTATACCAGCTGAAGGACCCCCGATCT
CAGGATAGTACTCTGTGCCTGTTCACCGACTTTGATAGTCAGATCAATGTGCCTAAAAC
CATGGAATCCGGAACTTTTATTACCGACAAGTGCGTGCTGGATATGAAAGCCATGGACA
GTAAGTCAAACGGCGCCATCGCTTGGAGCAATCAGACATCCTTCACTTGCCAGGATATC
TTCAAGGAGACCAACGCAACATACCCATCCTCTGACGTGCCCTGTGATGCCACCCTGA
CAGAGAAGTCTTTCGAAACAGACATGAACCTGAATTTTCAGAATCTGAGCGTGATGGGC
CTGAGAATCCTGCTGCTGAAGGTCGCTGGGTTTAATCTGCTGATGACACTGCGGCTGT
GGTCCTCATGA
SEQ ID NO: 34 (amino acid sequence for Vβ (VDJ) domain and constant domain of TCR
1C5.6):
MSNQVLCCVVLCFLGANTVDGGITQSPKYLFRKEGQNVTLSCEQNLNHDAMYWYRQDPG
QGLRLIYYSQIVNDFQKGDIAEGYSVSREKKESFPLTVTSAQKNPTAFYLCASSIAQGADTQ
YFGPGTRLTVLEDLNKVFPPEVAVFEPSEAEISHTQKATLVCLATGFFPDHVELSWWVNGK
EVHSGVSTDPQPLKEQPALNDSRYCLSSRLRVSATFWQNPRNHFRCQVQFYGLSENDEW
TQDRAKPVTQIVSAEAWGRADCGFTSVSYQQGVLSATILYEILLGKATLYAVLVSALVLMAM
VKRKDF
SEQ ID NO: 35 (amino acid sequence for Vβ (VDJ) domain of TCR 1C5.6 and constant domain
(murine)):
MSNQVLCCVVLCFLGANTVDGGITQSPKYLFRKEGQNVTLSCEQNLNHDAMYWYRQDPG
QGLRLIYYSQIVNDFQKGDIAEGYSVSREKKESFPLTVTSAQKNPTAFYLCASSIAQGADTQ
YFGPGTRLTVLEDLRNVTPPKVSLFEPSKAEIANKQKATLVCLARGFFPDHVELSWWVNGK
EVHSGVCTDPQAYKESNYSYCLSSRLRVSATFWHNPRNHFRCQVQFHGLSEEDKWPEGS
PKPVTQNISAEAWGRADCGITSASYHQGVLSATILYEILLGKATLYAVLVSGLVLMAMVKKK
NS
SEQ ID NO: 36 (nucleic acid sequence for Vβ (VDJ) domain and constant domain of TCR
1C5.6):
ATGAGCAACCAGGTGCTCTGCTGTGTGGTCCTTTGTTTCCTGGGAGCAAACACCGTGG
ATGGTGGAATCACTCAGTCCCCAAAGTACCTGTTCAGAAAGGAAGGACAGAATGTGAC
CCTGAGTTGTGAACAGAATTTGAACCACGATGCCATGTACTGGTACCGACAGGACCCA
GGGCAAGGGCTGAGATTGATCTACTACTCACAGATAGTAAATGACTTTCAGAAAGGAGA
TATAGCTGAAGGGTACAGCGTCTCTCGGGAGAAGAAGGAATCCTTTCCTCTCACTGTGA
CATCGGCCCAAAAGAACCCGACAGCTTTCTATCTCTGTGCCAGTAGTATTGCTCAGGGT
GCAGATACGCAGTATTTTGGCCCAGGCACCCGGCTGACAGTGCTCGAGGACCTGAACA
AGGTGTTCCCACCCGAGGTCGCTGTGTTTGAGCCATCAGAAGCAGAGATCTCCCACAC
CCAAAAGGCCACACTGGTGTGCCTGGCCACAGGCTTCTTCCCCGACCACGTGGAGCT
GAGCTGGTGGGTGAATGGGAAGGAGGTGCACAGTGGGGTCAGCACAGACCCGCAGC
CCCTCAAGGAGCAGCCCGCCCTCAATGACTCCAGATACTGCCTGAGCAGCCGCCTGA
GGGTCTCGGCCACCTTCTGGCAGAACCCCCGCAACCACTTCCGCTGTCAAGTCCAGTT
CTACGGGCTCTCGGAGAATGACGAGTGGACCCAGGATAGGGCCAAACCCGTCACCCA
GATCGTCAGCGCCGAGGCCTGGGGTAGAGCAGACTGTGGCTTTACCTCGGTGTCCTA
CCAGCAAGGGGTCCTGTCTGCCACCATCCTCTATGAGATCCTGCTAGGGAAGGCCACC
CTGTATGCTGTGCTGGTCAGCGCCCTTGTGTTGATGGCCATGGTCAAGAGAAAGGATT
TCTGA
SEQ ID NO: 37 (codon optimized nucleic acid sequence for Vβ (VDJ) domain of TCR 1C5.6
and constant domain (murine)):
ATGAGCAACCAGGTGCTGTGCTGCGTGGTGCTGTGCTTTCTTGGCGCTAACACAGTGG
ATGGAGGCATTACACAGAGCCCAAAGTACCTGTTTAGAAAGGAGGGGCAGAACGTGAC
ACTGAGCTGTGAGCAGAACCTGAACCACGATGCCATGTACTGGTACAGACAAGATCCA
GGACAGGGGCTGAGACTGATCTACTACAGTCAGATTGTGAACGATTTTCAGAAGGGAG
ATATTGCCGAGGGCTACAGCGTGTCTAGGGAGAAGAAGGAGTCTTTTCCACTGACAGT
GACTTCAGCCCAGAAGAACCCTACAGCCTTTTACCTGTGCGCTAGCAGCATTGCTCAG
GGCGCTGATACACAGTACTTTGGACCTGGGACAAGGCTGACAGTGCTGGAAGATCTAC
GTAACGTGACACCACCCAAAGTCTCACTGTTTGAGCCTAGCAAGGCAGAAATTGCCAAC
AAGCAGAAGGCCACCCTGGTGTGCCTGGCAAGAGGGTTCTTTCCAGATCACGTGGAGC
TGTCCTGGTGGGTCAACGGCAAAGAAGTGCATTCTGGGGTCTGCACCGACCCCCAGG
CTTACAAGGAGAGTAATTACTCATATTGTCTGTCAAGCCGGCTGAGAGTGTCCGCCACA
TTCTGGCACAACCCTAGGAATCATTTCCGCTGCCAGGTCCAGTTTCACGGCCTGAGTG
AGGAAGATAAATGGCCAGAGGGGTCACCTAAGCCAGTGACACAGAACATCAGCGCAGA
AGCCTGGGGACGAGCAGACTGTGGCATTACTAGCGCCTCCTATCATCAGGGCGTGCTG
AGCGCCACTATCCTGTACGAGATTCTGCTGGGAAAGGCCACCCTGTATGCTGTGCTGG
TCTCCGGCCTGGTGCTGATGGCCATGGTCAAGAAAAAGAACTCTTGA

### REFERENCES

1. Pont M J, Honders M W, Kremer A N, van Kooten C, Out C, Hiemstra P S, et al. Microarray Gene Expression Analysis to Evaluate Cell Type Specific Expression of Targets Relevant for Immunotherapy of Hematological Malignancies. PloS one. 2016; 11(5):e0155165.
2. Jahn L, Hombrink P, Hagedoorn R S, Kester M G, van der Steen D M, Rodriguez T, et al. TCR-based therapy for multiple myeloma and other B-cell malignancies targeting intracellular transcription factor BOB1. Blood. 2017; 129(10):1284-95.
3. Hombrink, P., C. Hassan, M. G. Kester, A. H. de Ru, C. A. van Bergen, H. Nijveen, J. W. Drijfhout, J. H. Falkenburg, M. H. Heemskerk, and P. A. van Veelen. 2013. Discovery of T cell epitopes implementing HLA-peptidomics into a reverse immunology approach. J. Immunol. 190:3869-3877.
4. Amir, A. L., D. M. van der Steen, M. M. van Loenen, R. S. Hagedoorn, B. R. de, M. D. Kester, A. H. de Ru, G. J. Lugthart, K. C. van, P. S. Hiemstra, I. Jedema, M. Griffioen, P. A. van Veelen, J. H. Falkenburg, and M. H. Heemskerk. 2011. PRAME-specific Allo-HLA-restricted T cells with potent antitumor reactivity useful for therapeutic T-cell receptor gene transfer. Clin. Cancer Res. 17:5615-5625.
5. Heemskerk, M. H., R. A. de Paus, E. G. Lurvink, F. Koning, A. Mulder, R. Willemze, J. J. van Rood, and J. H. Falkenburg. 2001. Dual HLA class I and class II restricted recognition of alloreactive T lymphocytes mediated by a single T cell receptor complex. Proc. Natl. Acad. Sci. U.S.A 98:6806-6811.
6. van Loenen, M.M., B. R. de, L. E. van, P. Meij, I. Jedema, J. H. Falkenburg, and M. H. Heemskerk. 2014. A Good Manufacturing Practice procedure to engineer donor virus-specific T cells into potent anti-leukemic effector cells. Haematologica 99:759-768.
7. Ruggieri L, et al., Hum Gene Ther. 1997; 8: 1611-1623.
8. WO2016/071758.
9. Mol Cell Proteomics, 2013; 12:1829
10. Scholten et al, Clin. Immunol. 119: 135, 2006
11. Govers et al, Trends Mol. Med. 16(2):11 (2010)
12. Sadelain et al, Cancer Discov., 3(4):388 (2013);
13. Harris and Kranz, Trends Pharmacol. Sci., 37(3):220 (2016)
14. Stone et al, Cancer Immunol. Immunother., 63(11): 1163 (2014)
15. U.S. Pat. No. 6,410,319
16. U.S. Pat. No. 7,446,191
17. U.S. Patent Publication No. 2010/065818
18. U.S. Pat. No. 8,822,647
19. WO 2014/031687
20. U.S. Pat. No. 7,514,537
21. Brentjens et al, 2007, Clin. Cancer Res. 73:5426
22. Monjezi et al., Leukemia 2017 31:186-194.
23. Coren et al., BioTechniques, 2015 58:135-139
24. Roth et al 2018 Nature vol 559; page 405
25. Szymczak et al., Nature Biotechnology 22, 589-594 (2004)
26. Jones et al., Human Gene Ther 2009 20: 630-640.
27. WO2004/106380
28. U.S. Publication No. 2004/0202657
29. U.S. Pat. No. 6,750,325
30. Willemsen et al, Gene Therapy 2000, 7:1369-77.
31. B S Jones, L S Lamb, F Goldman, A Di Stasi; Improving the safety of cell therapy products by suicide gene transfer. Front Pharmacol. (2014) 5:254.
32. Sambrook et al (1989) Molecular Cloning, A Laboratory Manual, Cold Spring Harbor Laboratory, Cold Spring Harbor, N.Y
33. Ausubel et al (1987) Current Protocols in Molecular Biology, John Wiley and Sons, Inc., NY
34. Cohen et al (1972) Proc. Natl. Acad. Sci. USA 69, 2110
35. Luchansky et al (1988) Mol. Microbiol. 2, 637-646
36. Morton, L. T., Reijmers, R. M., Wouters, A. K., Kweekel, C., Remst, D. F. G., Pothast, C. R., Falkenburg, J. H. F. & Heemskerk, M. H. M. (2020) Simultaneous Deletion of Endogenous TCRαβ for TCR Gene Therapy Creates an Improved and Safe Cellular Therapeutic, Mol Ther. 28, 64-74.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. An isolated nucleic acid composition that encodes a Bob1 antigen-specific binding protein having a TCR α chain variable (Vα) domain and a TCR β chain variable (Vβ) domain, the composition comprising:
(a) a nucleic acid sequence that encodes a TCR Vα domain comprising a CDR3 amino acid sequence having at least 80% sequence identity to SEQ ID NO:12, or a functional fragment thereof; and
(b) a nucleic acid sequence that encodes a TCR Vβ domain comprising a CDR3 amino acid sequence having at least 80% sequence identity to SEQ ID NO: 21, or a functional fragment thereof.

2. The nucleic acid composition of claim 1, wherein the Bob1 antigen comprises the amino acid sequence LPHQPLATY.

3. The nucleic add composition of any preceding claim, wherein the encoded binding protein is capable of specifically binding to a LPHQPLATY:HLA-B*35:01 complex.

4. The nucleic acid composition of any preceding claim, wherein the nucleic acid sequence is codon optimised for expression in a host cell, optionally wherein the host cell is a human cell.

5. The nucleic acid composition of any preceding claim, wherein:
(i) the CDR3 of the Vα domain comprises or consists of the amino acid sequence of SEQ ID NO: 12, and
(ii) the CDR3 of the Vβ domain comprises or consists of the amino acid sequence of SEQ ID NO:21.

6. The nucleic acid composition of claim 5, wherein:
(i) the CDR3 of the Vα domain is encoded by a nucleic acid sequence comprising the sequence of SEQ ID NO: 13 or SEQ ID NO:14, or a derivative thereof; and/or
(ii) the CDR3 of the Vβ domain is encoded by a nucleic acid sequence comprising the sequence of SEQ ID NO: 22 or SEQ ID NO:23, or a derivative thereof.

7. The nucleic acid composition of any preceding claim, wherein:
(i) the Vα domain comprises an amino acid sequence having at least 80% sequence identity to, comprising, or consisting of, SEQ ID NO: 24; and/or
(ii) the Vβ domain comprises an amino acid sequence having at least 80% sequence identity to, comprising, or consisting of, SEQ ID NO: 27.

8. The nucleic acid composition of claim 7, wherein:
(i) the Vα domain is encoded by a nucleic acid sequence comprising the sequence of SEQ ID NO: 25 or SEQ ID NO: 26; and/or
(ii) the Vβ domain is encoded by a nucleic acid sequence comprising the sequence of SEQ ID NO: 28 or SEQ ID NO:29.

9. The nucleic acid composition of any preceding claim, further comprising a TCR α chain constant domain and/or a TCR β chain constant domain.

10. The nucleic acid composition of any preceding claim, wherein the encoded binding protein comprises a TCR, an antigen binding fragment of a TCR, or a chimeric antigen receptor (CAR).

11. The nucleic acid composition of claim 10, wherein the antigen binding fragment of a TCR is a single chain TCR (scTCR) or a chimeric TCR dimer in which the antigen binding fragment of the TCR is linked to an alternative transmembrane and intracellular signalling domain.

12. A vector system comprising a nucleic acid composition according to any one of claims 1 to 11.

13. The vector system of claim 12, wherein the vector is a plasmid, a viral vector, or a cosmid, optionally wherein the vector is selected from the group consisting of a retrovirus, lentivirus, adeno-associated virus, adenovirus, vaccinia virus, canary poxvirus, herpes virus, minicircle vector and synthetic DNA or RNA.

14. A modified cell comprising a nucleic acid composition according to any of claims 1 to 11, or a vector system according to claim 12 or 13.

15. The modified cell of claim 14, wherein the modified cell is selected from the group consisting of a CD8 T cell, a CD4 T cell, an NK cell, an NK-T cell, a gamma-delta T cell, a hematopoietic stem cell, an inducible pluripotent stem cell, a progenitor cell, a T cell line and a NK-92 cell line.

16. The modified cell of claim 14 or 15, wherein the modified cell is a human cell.

17. A pharmaceutical composition comprising a nucleic acid composition according to any of claims 1 to 11, a vector system according to claim 12 or 13, or a modified cell according to any of claims 14 to 16, and a pharmaceutically acceptable excipient, adjuvant, diluent and/or carrier.

18. A pharmaceutical composition according to claim 17 for use in inducing or enhancing an immune response in an HLA-B*35:01 positive human subject diagnosed with a hyperproliferative disease or condition.

19. A pharmaceutical composition according to claim 17 for use in stimulating a cell mediated immune response to a target cell population or tissue in an HLA-B*35:01 positive human subject.

20. A pharmaceutical composition according to claim 17 for use in providing anti-tumor immunity to an HLA-B*35:01 positive human subject.

21. A pharmaceutical composition according to claim 17 for use in treating an HLA-B*35:01 positive human subject having a disease or condition associated with an elevated level of Bob1.

22. The pharmaceutical composition for use according to any of claims 18 to 21 wherein the subject has at least one tumor.

23. The pharmaceutical composition for use according to any of claims 18 to 22 wherein the subject has been diagnosed with a B cell malignancy or multiple myeloma, optionally wherein the B cell malignancy is a B cell lymphoma or a B cell leukemia, further optionally wherein the B cell malignancy is selected from the group consisting of mantle cell lymphoma, acute lymphoblastic leukemia, chronic lymphocytic leukemia, follicular lymphoma and large B cell lymphoma.

24. A method of generating a binding protein that is capable of specifically binding to a peptide containing a Bob1 antigen and does not bind to a peptide that does not contain the Bob1 antigen, comprising contacting a nucleic acid composition according to any of claims 1 to 11 with a cell under conditions in which the nucleic acid composition is incorporated and expressed by the cell.

25. The method of claim 24, wherein the method is ex vivo.

26. An isolated nucleic acid sequence comprising or consisting of the nucleotide sequence of any one of SEQ ID NOs: 13, 14, 22, 23, 25, 26, 28, 29, 32, 33, 36 or 37.

27. An isolated nucleic acid sequence comprising or consisting of the nucleotide sequence of any one of SEQ ID NOs: 13, 14, 22, 23, 25, 26, 28, 29, 32, 33, 36 or 37 for use in therapy.
